SEQUENCE LISTING (8 sequences)

SEQ ID NO: 1 - DNA, 24 bp, Artificial sequence
  Synthetic primer
gggtgtacctcttaatgccaattc

SEQ ID NO: 2 - DNA, 20 bp, Artificial sequence
  Synthetic primer
tctgtcctgtctccgccaat

SEQ ID NO: 3 - DNA, 18 bp, Artificial sequence
  Synthetic primer
acccctgcgcaaaatcgt

SEQ ID NO: 4 - DNA, 19 bp, Artificial sequence
  Synthetic primer
cacattggcacccgcaatc

SEQ ID NO: 5 - DNA, 20 bp, Artificial sequence
  Synthetic primer
gaggaacgagaagaggcttg

SEQ ID NO: 6 - DNA, 25 bp, Artificial sequence
  Synthetic primer
acttcctcaaggaacaacattgcca

SEQ ID NO: 7 - DNA, 24 bp, Artificial sequence
  Synthetic primer
agatcaacttctgtcatccagcaa

SEQ ID NO: 8 - DNA, 29 bp, Artificial sequence
  Synthetic primer
ttctgcacatcataattaggagtatcaat

(19) United States
(12) Patent Application Publication
(10) Pub. No.: US 2024/0041805 A1
(43) Pub. Date: Feb. 8, 2024

(54) PHARMACEUTICAL COMPOSITIONS OF NICLOSAMIDE AND A PROTEIN

(71) Applicant: BOARD OF REGENTS, THE UNIVERSITY OF TEXAS SYSTEM, Austin, TX (US)

(72) Inventors: Hugh D.C. SMYTH, Austin, TX (US); Ashlee BRUNAUGH, Austin, TX (US); Zachary N. WARNKEN, Austin, TX (US); Hyo-Jong SEO, Austin, TX (US)

(21) Appl. No.: 18/044,877

(22) PCT Filed: Sep. 17, 2021
(86) PCT No.: PCT/US2021/050848

§ 371 Date: Mar. 10, 2023

Related U.S. Application Data
(60) Provisional application No. 63/079,674, filed on Sep. 17, 2020.

(51) Int. Cl. A61K 31/167; A61K 47/26; A61K 38/47; A61K 9/00; A61P 31/14; A61P 31/04; A61K 47/18; A61K 47/14

(52) U.S. Cl. CPC A61K 31/167; A61K 47/26; A61K 38/47; A61K 9/0075; A61P 31/14; A61P 31/04; A61K 47/183; A61K 47/14; C12Y 302/01017

(57) ABSTRACT

The present disclosure provides pharmaceutical compositions of niclosamide and a protein that may be administered via inhalation These compositions may be used in nebulized, nasal, and inhaler formats. These compositions may be used to treat one or more diseases or disorders such as a viral infection or cancer.

This application claims the benefit of priority to U.S. Provisional Application No. 63/079,674, filed on Sep. 17, 2020, the entire contents of which are hereby incorporated by reference.

This invention was made with government support under Grant no. F31 HL146178 awarded by the National Institutes of Health. The government has certain rights in the invention.

### BACKGROUND

### 1. Field

The present disclosure relates generally to the field of pharmaceuticals and pharmaceutical manufacture. More particularly, it concerns compositions and methods of preparing a pharmaceutical composition comprising niclosamide and a protein.

### 2. Description of Related Art

The World Health Organization (WHO) declared the Coronavirus Disease 2019 (COVID-19) outbreak a pandemic in March 2020 (World Health Organization, 2020). A rapid and continuing rise in cases worldwide followed this declaration which has overwhelmed healthcare systems (Li et al., 2020; Miller et al., 2020), and outbreaks are expected to continue potentially into 2025(Kissler et al., 2020). The causative agent of the COVID-19 pandemic is SARS-CoV-2, which is related to other pandemic-causing coronaviruses, the Severe Acute Respiratory Syndrome-associated coronavirus (SARS-CoV) and the Middle East respiratory syndrome-related coronavirus (MERS-CoV).(Wu et al., 2004; Peeri et al., 2020). The current COVID-19 pandemic has killed more people than the SARS and MERS pandemics combined (Gurwitz, 2020) and at present there are no available vaccines for coronaviruses (Walls et al., 2020; Tortorici et al., 2019). Effective treatment options for COVID-19 are thus desperately needed.

To successfully quell the pandemic, it is necessary that treatments can be made rapidly available and low-cost to patients and clinicians worldwide. This requirement has resulted in the strategy of assessing drugs previously used in SARS and MERS for utility against SARS-CoV-2. (Wang et al., 2020) One particularly promising agent is the drug called niclosamide (NIC), which is an FDA-approved is an FDA approved anthelmintic drug that is listed as an Essential Medicine by the WHO (Barbosa et al., 2019) and has been in use for over 60 years. NIC has been proposed as a candidate for re-purposing against a broad spectrum of indications, including multiple oncology, antiviral, and antibacterial targets (Xu et al., 2020; Li et al., 2014; Chen and Mook, 2018; Tam et al., 2018). The main feature that enables these broad-spectrum therapeutic effects are the physical chemistry and protonophoric activity of the molecule itself, rather than specific ligand-receptor interactions. (Fonseca et al., 2012) Niclosamide has been shown to be highly effective against coronaviruses including MERS and SARS-CoV-2 (Gassen et al., 2019) and has been proposed as a potential therapeutic candidate for the current COVID-19 pandemic. The drug's non-specific mechanism of action also enables broad activity against both gram-positive and gram-negative bacteria, which would offer promising protection against secondary bacterial pneumonias associated with COVID-19. Furthermore, NIC has been noted to inhibit inflammatory cytokine release in lung tissues in vivo (Cabrita et al., 2019), which may provide an important protective effect against cytokine storm and acute respiratory distress syndrome (ARDS), one of the most feared consequences of COVID-19.

The main limitation of NIC is its poor solubility in water, which is reported at 1.6 mg/L(PubChem CID=4477, 2020). This makes systemic absorption of the drug by the oral route of administration exceedingly difficult. Administration of the existing FDA-approved chewable tablet formulation was found to result in inadequate systemic concentrations for inhibiting SARS-CoV-2 replication.(Jeon et al., 2020; Schweizer et al., 2018) As an alternative, direct delivery of niclosamide the lung could overcome the limitations of the oral NIC formulation by generating high drug concentrations at the site of infection. Costabile et al previously developed an inhalable dry powder consisting of NIC nanocrystals embedded in mannitol particles.(Costabile et al., 2015) However, these particles required a high amount of polysorbate 80 to ensure production of a stable suspension (10% w/w to NIC) which is beyond what is currently approved by the FDA for the oral inhalation route (Inactive Ingredients Database, 2019). Furthermore, the utilization of mannitol as the carrier system may induce bronchospasm and cough (Kanth et al., 2018; Koskela et al., 2005) which may contribute to increased risk of spread of SARS-CoV-2 through respiratory droplets.

An additional considerable challenge in the treatment of COVID-19 is the variable presentation of illness. Patients may act as asymptomatic carriers of the virus or develop severe pneumonias and acute respiratory disease (Lai et al., 2020), which can result in the requirement for mechanical ventilation. In the case of ventilated patients, a nebulizer is often used to delivery aerosolized drug to the lungs. However, for treatment of asymptomatic carriers or in developing regions with reduced access to clean water sources, nebulizer-based therapy may present an undue burden and reduce therapy compliance. For these populations, dry powder inhaler (DPI) or nasal spray, or a combination of both, would be the preferred option based upon the rapid administration time and ease of use and could also potentially be utilized as a prophylactic therapy in high risk populations such as healthcare workers and first responders.

For these reasons, there remains a need to develop pharmaceutical compositions of niclosamide that are amendable to use in a variety of different formats.

### SUMMARY OF THE INVENTION

In some aspects, the present disclosure provides pharmaceutical compositions comprising niclosamide and a protein. In some embodiments, the protein is a protein that is positively charged at physiological pH. In some embodiments, the protein is an immunomodulating protein. In some embodiments, the protein is therapeutically active against a coronavirus. In some embodiments, the protein is therapeutically active against SARS-CoV-2. In some embodiments, the protein is lysozyme, such as a human lysozyme. In some embodiments, the lysozyme is a recombinant lysozyme such as a recombinant human lysozyme. In some embodiments, the protein has been modified to reduce its degradation in vivo.

In some embodiments, the pharmaceutical composition further comprises an excipient. In some embodiments, the excipient is a sugar or a sugar derivative. In further embodiments, the excipient is a sugar. In still further embodiments, the sugar is a disaccharide, such as sucrose. In some embodiments, the excipient is a compound with a hydrophobic component and a PEG or polypropylene glycol component. In some embodiments, the hydrophobic component is a fatty acid. In some embodiments, the PEG or polypropylene glycol component is a PEGylated polysorbate. In some embodiments, the excipient is Tween®. In some embodiments, the excipient is Tween® 80. In some embodiments, the pharmaceutical composition comprises two or more excipients. In some embodiments, the pharmaceutical composition comprises a first excipient and a second excipient. In some embodiments, the two or more excipients are a disaccharide and PEGylated polysorbate. In some embodiments, the two or more excipients are sucrose and Tween® 80.

In some embodiments, niclosamide comprises from about 0.1% w/w to about 5% w/w of the pharmaceutical composition. In further embodiments, niclosamide comprises from about 0.2% w/w to about 2.5% w/w of the pharmaceutical composition. In still further embodiments, niclosamide comprises from about 0.25% w/w to about 1.25% w/w of the pharmaceutical composition. In yet further embodiments, niclosamide comprises 0.6% w/w to 0.8% w/w of the pharmaceutical composition. In some embodiments, the protein comprises from about 40% w/w to about 95% w/w of the pharmaceutical composition. In further embodiments, the protein comprises from about 50% w/w to about 90% w/w of the pharmaceutical composition. In still further embodiments, the protein comprises from about 55% w/w to about 85% w/w of the pharmaceutical composition. In yet further embodiments, the protein comprises from about 60% w/w to about 80% w/w of the pharmaceutical composition.

In some embodiments, the first excipient comprises from about 5% w/w to about 60% w/w of the pharmaceutical composition. In further embodiments, the first excipient comprises from about 10% w/w to about 50% w/w of the pharmaceutical composition. In still further embodiments, the first excipient comprises from about 15% w/w to about 45% w/w of the pharmaceutical composition. In yet further embodiments, the first excipient comprises from about 20% w/w to about 40% w/w of the pharmaceutical composition. In some embodiments, the second excipient comprises from about 0.001% w/w to about 2.5% w/w of the pharmaceutical composition. In further embodiments, the second excipient comprises from about 0.01% w/w to about 1.0% w/w of the pharmaceutical composition. In still further embodiments, the second excipient comprises from about 0.025% w/w to about 0.5% w/w of the pharmaceutical composition. In yet further embodiments, the second excipient comprises from about 0.05% w/w to about 0.25% w/w of the pharmaceutical composition.

In some embodiments, the pharmaceutical composition has a X90 diameter from about 1.0 μm to about 15 μm as a dry powder. In further embodiments, the X90 diameter is from about 2.0 μm to about 10 μm as a dry powder. In still further embodiments, the X90 diameter is from about 4.0 μm to about 8.0 μm as a dry powder. In yet further embodiments, the X90 diameter is from about 5.0 μm to about 7.0 μm as a dry powder. In some embodiments, X90 diameter is measured for an aqueous solution. In further embodiments, the aqueous solution is saline. In some embodiments, the pharmaceutical composition has a zeta potential from about −25 to about 10 of a 10 mg/mL concentration solution reconstituted in water. In further embodiments, the zeta potential is from about −15 to about 5. In still further embodiments, the zeta potential is from about −5 to about 5. In yet further embodiments, the zeta potential is from about 0 to about 5. In some embodiments, the zeta potential is positive. In some embodiments, the pharmaceutical composition is stored in a container to protect from UV light.

In some embodiments, the pharmaceutical composition is formulated for administration via inhalation. In some embodiments, the pharmaceutical composition is formulated for aerosol administration to form an aerosol pharmaceutical composition. In some embodiments, the aerosol pharmaceutical composition has been formulated into an inhaler. In some embodiments, the inhaler is a dry powder inhaler. In some embodiments, the inhaler is a disposable inhaler. In some embodiments, the inhaler is a TwinCaps or Orbital dry powder inhaler.

In some embodiments, the aerosol pharmaceutical composition comprises an emitted fraction of greater than 40% for an inhaler loaded with 60 mg of the pharmaceutical composition with about 0.70% w/w of niclosamide. In further embodiments, the emitted fraction is greater than 50%. In still further embodiments, the emitted fraction is greater than 60%. In some embodiments, the aerosol pharmaceutical composition comprises an emitted dose from about 200 μg to about 500 μg for an inhaler loaded with 60 mg of the pharmaceutical composition with about 0.70% w/w of niclosamide. In further embodiments, the emitted dose is from about 250 μg to about 400 μg of niclosamide. In still further embodiments, the emitted dose is from about 250 μg to about 350 μg of niclosamide.

In some embodiments, the aerosol pharmaceutical composition has a fine particle fraction of less than 5 μm of greater than 25%. In further embodiments, the fine particle fraction is greater than 35%. In still further embodiments, the fine particle fraction is greater than 40%. In some embodiments, the aerosol pharmaceutical composition has a fine particle dose of less than 5 μm is from about 50 μg to about 300 μg of niclosamide. In further embodiments, the fine particle fraction is from about 75 μg to about 200 μg of niclosamide. In still further embodiments, the fine particle fraction is from about 100 μg to about 150 μg of niclosamide. In some embodiments, the aerosol pharmaceutical composition has a fine particle fraction of less than 3 μm of greater than 15%. In further embodiments, the fine particle fraction is greater than 20%. In still further embodiments, the fine particle fraction is greater than 25%. In some embodiments, the aerosol pharmaceutical composition has a fine particle dose of less than 3 μm is from about 10 μg to about 150 μg of niclosamide. In further embodiments, the fine particle dose is from about 50 μg to about 125 μg of niclosamide. In still further embodiments, the fine particle dose is from about 75 μg to about 100 μg of niclosamide. In some embodiments, the aerosol pharmaceutical composition comprises a mean median aerodynamic diameter from about 2.0 μm to about 10.0 μm. In further embodiments, the mean median aerodynamic diameter is from about 4.0 μm to about 8.0 μm. In still further embodiments, the mean median aerodynamic diameter is from about 6.0 μm to about 7.0 μm.

In some embodiments, the pharmaceutical composition is formulated for nebulization to form a nebulized pharmaceutical composition. In some embodiments, the pharmaceutical composition has been formulated into a nebulized aqueous solution. In some embodiments, the pharmaceutical composition has been reconstituted in water. In some embodiments, the water is saline. In some embodiments, the saline is half normal saline. In some embodiments, the nebulized pharmaceutical composition comprises a concentration of the pharmaceutical composition from about 1 mg/mL to about 1 g/mL. In further embodiments, the concentration of the pharmaceutical composition is from about 5 mg/mL to about 500 mg/mL. In still further embodiments, the concentration of the pharmaceutical composition is from about 10 mg/mL to about 250 mg/mL. In some embodiments, the nebulized pharmaceutical composition has a fine particle fraction of less than 5 μm of greater than 45%. In further embodiments, the fine particle fraction is greater than 50%. In still further embodiments, the fine particle fraction is greater than 55%. In some embodiments, the nebulized pharmaceutical composition has a fine particle fraction of less than 3 μm of greater than 25%. In further embodiments, the fine particle fraction is greater than 30%. In still further embodiments, the fine particle fraction is greater than 35%. In some embodiments, the nebulized pharmaceutical composition comprises a mean median aerodynamic diameter from about 2.0 μm to about 10.0 μm. In further embodiments, the mean median aerodynamic diameter is from about 4.0 μm to about 8.0 μm. In still further embodiments, the mean median aerodynamic diameter is from about 5.5 μm to about 6.5 μm.

In some embodiments, the pharmaceutical composition is formulated for nasal administration as a nasal pharmaceutical composition. In some embodiments, the nasal pharmaceutical composition is suspended in water. In some embodiments, the water is saline. In some embodiments, the saline is 0.45% w/v sodium chloride solution. In some embodiments, the nasal pharmaceutical composition has been formulated for actuation using a nasal spray device. In some embodiments, the nasal pharmaceutical composition comprises a concentration of the pharmaceutical composition from about 1 mg/mL to about 1 g/mL. In some embodiments, the concentration of the pharmaceutical composition is from about 2.5 mg/mL to about 500 mg/mL. In further embodiments, the concentration of the pharmaceutical composition is from about 5 mg/mL to about 100 mg/mL. In some embodiments, the nasal pharmaceutical composition has a plume angle from about 20° to about 80° when emitted from a VP7 pump spray device. In some embodiments, the plume angle is from about 30° to about 70°. In some embodiments, the plume angle is from about 40° to about 60°.

In some embodiments, the nasal pharmaceutical composition has a spray area from about 100 mm² to about 1000 mm² when measured from at 2 cm from the nasal spray device. In further embodiments, the spray area is from about 250 mm² to about 750 mm². In still further embodiments, the spray area is from about 400 mm² to about 600 mm². In some embodiments, the nasal pharmaceutical composition has a minimum spray diameter from about 10 mm to about 50 mm In further embodiments, the minimum spray diameter is from about 15 mm to about 30 mm In still further embodiments, the minimum spray diameter is from about 20 mm to about 25 mm In some embodiments, the nasal pharmaceutical composition has a maximum spray diameter from about 10 mm to about 50 mm In further embodiments, the maximum spray diameter is from about 15 mm to about 30 mm In still further embodiments, the maximum spray diameter is from about 22.5 mm to about 30 mm

In some embodiments, the nasal pharmaceutical composition has a spray area from about 500 mm² to about 5000 mm² when measured from at 5 cm from the nasal spray device. In further embodiments, the spray area is from about 750 mm² to about 4500 mm². In still further embodiments, the spray area is from about 1000 mm² to about 3000 mm². In some embodiments, the nasal pharmaceutical composition has a minimum spray diameter from about 20 mm to about 100 mm. In further embodiments, the minimum spray diameter is from about 30 mm to about 75 mm. In still further embodiments, the minimum spray diameter is from about 35 mm to about 60 mm. In some embodiments, the nasal pharmaceutical composition has a maximum spray diameter from about 25 mm to about 150 mm. In further embodiments, the maximum spray diameter is from about 35 mm to about 100 mm. In still further embodiments, the maximum spray diameter is from about 45 mm to about 65 mm.

In other aspects, the present disclosure provides methods of preparing a pharmaceutical composition of the present disclosure comprising:
(A) admixing niclosamide and the protein with a solvent to obtain a pharmaceutical mixture;
(B) subjecting the pharmaceutical mixture to spray drying to obtain the pharmaceutical composition.
   In some embodiments, the pharmaceutical mixture further comprises an excipient. In some embodiments, the pharmaceutical mixture comprises two excipients. In some embodiments, the pharmaceutical mixture comprises a step of admixing a first excipient. In some embodiments, the pharmaceutical mixture comprises a step of admixing a second excipient. In some embodiments, the first excipient is admixed before the second excipient.

In some embodiments, the niclosamide is micronized niclosamide. In some embodiments, the niclosamide has a X50 diameter from about 1.5 μm to about 2.5 μm. In further embodiments, the niclosamide has a X90 diameter from about 3.5 μm to about 4.5 μm.

In some embodiments, the niclosamide has a needle morphology. In some embodiments, the either the first excipient or second excipient is added as an aqueous solution. In some embodiments, the solvent is water. In some embodiments, the water further comprises a buffer. In some embodiments, the buffer is a histidine buffer. In some embodiments, the histidine buffer is a 0.175 mg/mL histidine buffer.

In some embodiments, the spray drying comprises an inlet temperature from about 80° C. to about 180° C. In further embodiments, the inlet temperature is from about 90° C. to about 160° C. In still further embodiments, the inlet temperature is from about 100° C. to about 140° C. In yet further embodiments, the inlet temperature is about 130° C. In some embodiments, the spray drying comprises using an atomization gas. In some embodiments, the atomization gas has a flow rate from about 5 L/min to about 50 L/min. In further embodiments, the flow rate is from about 15 L/min to about 35 L/min. In still further embodiments, the flow rate is 22.9 L/min In some embodiments, the spray drying comprises a feed flow rate from about 0.05 mL/min to about 50 mL/min. In further embodiments, the feed flow rate is from about 0.1 mL/min to about 40 mL/min. In still further embodiments, the feed flow rate is about 1 mL/min. In some embodiments, the spray drying comprises using a dehumidifier unit. In some embodiments, the spray drying further comprises using a 2 fluid pneumatic nozzle.

In still other aspects, the present disclosure provides pharmaceutical compositions prepared using the methods of the present disclosure.

In yet other aspects, the present disclosure provides methods of treating a disease or disorder in a patient comprising administering a pharmaceutical composition of the present disclosure to the patient in a therapeutically effective amount. In some embodiments, the disease or disorder is a microbial infection. In some embodiments, the microbial infection is a viral infection. In some embodiments, the viral infection is an infection of a coronavirus. In some embodiments, the coronavirus is MERS-Cov, SARS-Cov1, or SARS-Cov2 (COVID-19). In some embodiments, the viral infection is influenza. In some embodiments, the viral infection is Zika. In some embodiments, the viral infection is a respiratory syncytial virus. In some embodiments, the microbial infection is hemorrhagic fever. In some embodiments, the hemorrhagic fever is Ebola and Lassa fever. In some embodiments, the viral infection is HIV. In some embodiments, HIV presents with tuberculosis. In some embodiments, the microbial infection is a flatworm infection. In some embodiments, the flatworm infection is Schistosomiasis or complication from Schistosomiasis. In some embodiments, the Schistosomiasis is acute pulmonary Schistosomiasis. In some embodiments, the complication from schistosomiasis is schistosomiasis associated pulmonary hypertension. In some embodiments, the microbial infection is bacterial infection. In some embodiments, the bacterial infection is an infection of enterococci, pseudomonas aeruginosa, staphylococcus aureus, or clostridium difficile. In some embodiments, the bacterial infection is an infection of a bacteria resistant to one or more antibiotics. In some embodiments, the infection is an infection of a bacteria resistant to vancomycin or methicillin.

In some embodiments, the disease or disorder is cancer. In some embodiments, the cancer is lung cancer, glioblastoma, or prostate cancer. In some embodiments, the prostate cancer is a castration resistant prostate cancer. In some embodiments, the disease or disorder is diabetes. In some embodiments, the methods further comprise a second active agent. In some embodiments, the second active agent is an anti-inflammatory. In some embodiments, the second active agent is clofazimine. In some embodiments, the second active agent is anti-microbial. In some embodiments, the second active agent is chloroquine, hydroxychloroquine, thalidomide, plasminogen, colistin, or polymyxin B. In some embodiments, the second active agent is chemotherapeutic agent. In some embodiments, the chemotherapeutic agent is abiraterone, enzalutamide, or bicalutamide. In some embodiments, the active agent is inhaled to the lungs. In some embodiments, the active agent is inhaled into the lungs and the stomach.

In other aspects, the present disclosure provides methods of reducing lung inflammation in a patient comprising administering a pharmaceutical composition of the present disclosure to the patient in a therapeutically effective amount. In some embodiments, the lung inflammation is associated with a viral infection. In some embodiments, the lung inflammation is associated with a bacterial infection. In some embodiments, the method results in a reduced production of IL-6. In some embodiments, the method results in a reduced production of TNFα. In some embodiments, the method results in an increased production of IL-10. In some embodiments, the pharmaceutical composition is administered more than once.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows the particle size distributions of the dry powders and reconstituted suspensions produced using examples listed in Table 1.

FIG. 2 shows the particle size distribution of suspensions generated from spray dried NIC-hLYS powder (diamonds) and NIC-BSA powder (circles).

FIGS. 3A-3F show NIC-hLYS exhibited enhanced anti-viral potency compared to NIC particles alone (NIC-M) in both MERS-CoV (FIG. 3A) and SARS-CoV-2 (FIG. 3B) infected cells. NIC-hLYS particles exhibited significantly higher inhibition of viral replication in SARS-CoV-2 infected Vero cells compared to an equivalent dose of solubilized NIC (NIC-DMSO), which indicates that improvements in solubility alone may not be sufficient to achieve maximal antiviral efficacy (FIG. 3C). Similar dose response profiles were noted for NIC-hLYS in both MERS-CoV (FIG. 3D) and SARS-CoV-2 (FIG. 3E), where an initial drop in activity is preceded by a sharp rise in activity. This profile was also noted in inhibitory assays for S. aureus. HLYS alone exhibits some antiviral activity which was pronounced at 48 hours post dosing than 24 hours (FIG. 3F). Data presented as mean+SEM (n=3). ***p<0.001, ****p<0.0001 using two-way ANOVA with Tukey's multiple comparisons test.

FIGS. 4A-4G show the once daily intranasal administration of NIC-hLYS particles suspended in 0.45% NaCl resulted in 43% survival in a lethal MERS-CoV infection (FIG. 4B) and produced a statistically significant decrease in lung viral titers at the highest dose tested (FIG. 4C). The viral particles obtained from lung and brain homogenates of surviving animals did not produce CPE when administered to Vero E6 cells, indicated that they were no longer active. Compared to lung tissue of uninfected mice (FIG. 4D), hDPP4-TG mice infected with MERS-CoV exhibited severe interstitial pneumonia on Day 6 of infection (FIG. 4E), mice treated with NIC-hLYS exhibited milder interstitial pneumona by Day 6 of treatment (FIG. 4F), which was further reduced by Day 14 (FIG. 4G). Data are presented as mean+SEM (n=3). *p<0.05, using two-way ANOVA with Dunnet's multiple comparisons test.

FIGS. 5A-5G show once daily intranasal administration of NIC-hLYS particles suspended in 0.45% NaCl resulted in 30% survival in a lethal SARS-CoV-2 infection (FIG. 5B) and produced a statistically significant decrease in lung viral titers after 10 days of dosing (FIG. 5C). The viral particles obtained from lung and brain homogenates of surviving animals did not produce CPE when administered to Vero E6 cells, indicated that they were no longer active. Compared to lung tissue of uninfected mice (FIG. 5D), infection with SARS-CoV-2 resulted in the development of interstitial pneumonia without treatment (FIG. 5E). By Day 6 of treatment with 240 μg/kg NIC, interstitial pneumonia was notably reduced (FIG. 5F) and further resolved by Day 14 (FIG. 5G). Data are presented as mean+SEM (n=3). *p<0.05, using two-way ANOVA with Dunnet's multiple comparisons test.

FIGS. 6A-6F show NIC-hLYS resulted in 50% inhibition of the S. aureus Mu50 strain at lower doses than several other NIC formulations tested, included solubilized NIC (FIGS. 6A-6C). Compared to NIC particles alone, 100% inhibition was achieved at a lower dose (FIG. 6B). Data presented as mean+SEM (n=6), **p<0.01,****p<0.0001, using two-way ANOVA with Dunnet's multiple comparisons test. NIC-hLYS significantly reduced production of the inflammatory cytokines IL-6 (FIG. 6D) and TNF-α (FIG. 6E) in THP-1 macrophages stimulated with 10 ng/mL lipopolysaccharide (LPS), though a significant increase in IL-1β production was noted at the highest dose tested compared to both the untreated control and NIC-BSA formulation, which may point towards the role of hLYS in inducing production of this cytokine. Data presented as mean+SEM (n=3), *p<0.05, ** p<0.01, ***p<0.001, ****p<0.0001 using one-way ANOVA with Sidak's multiple comparisons test.

FIG. 7A-7I show micronized niclosamide (FIG. 7A) was embedded in a matrix of human lysozyme and stabilizers using spray drying (FIG. 7B). This system was developed 25 as an alternative to traditional lactose-based carrier systems (FIG. 7C) and enabled the targeted respiratory delivery of NIC as a powder via DPI or a reconstituted suspension via nebulizer or nasal spray. The optimized formulation exhibited a size distribution that was appropriate for inhalation (i.e., geometric median diameter <5 μm) in both the dry powder state as well as when reconstituted using water or 0.45% NaCl (FIG. 7D). Similar effects could not be achieved when a negatively charged protein, bovine serum albumin, was substituted in the formulation for the positively charged hLYS (FIG. 7E). Though hLYS is surface active, it appeared to only slightly enhance the dissolution of NIC compared to NIC particles blended with lactose (FIG. 7F). A respirable droplet size distribution could be achieved with multiple different reconstituted concentrations when nebulized using the Aerogen Solo (FIG. 7G). These concentrations resulted in no aggregation to the lysozyme component. Efficient aerosol delivery was achieved with both the nebulizer and disposable DPI, with ˜50% of the emitted dose being of an appropriate size for lung deposition. This was significantly improved compared to a traditional lactose carrier particle system (FIG. 7H). Reproducible plume geometry could be achieved using a variety of reconstituted concentrations when actuated using the Aptar device (FIG. 7I). Data is presented as mean+SEM (n=3). Data is presented as mean+SEM (n=3). *p<0.05, using two-way ANOVA with Tukey's multiple comparisons test (comparisons of DPIs presented only).

FIGS. 8A-8F show the spray drying (FIG. 8B) did not result in additional formation of soluble protein aggregates compared to the unprocessed control (FIG. 8A). NIC-hLYS reconstituted at a concentration of 25 mg/mL (FIG. 8C), 50 mg/mL (FIG. 8D), 75 mg/mL (FIG. 8E), and 100 mg/mL (FIG. 8F) was nebulized over the course of 2 minutes from an Aerogen Solo vibrating mesh nebulizer. This process did not result in additional aggregation of lysozyme compared to the unprocessed control.

FIG. 9 shows the effect of varying doses of human lysozyme on the viability of A549-THP1 co-cultures.

FIG. 10 shows the effects of NIC on viability of A549-THP1 cultures (lung carcinoma model) in combination with varying doses of lysozyme. Data presented as mean+SEM (n=3). *p<0.05, **p<0.01 using ANOVA with Tukey's multiple comparisons test.

FIG. 11 shows the change in viral inhibition as a function of dosage.

FIG. 12A shows the change in viral counts as a function of dosage after 24 hours.

FIG. 12B shows the change in viral counts as a function of dosage after 48 hours.

### DESCRIPTION OF ILLUSTRATIVE EMBODIMENTS

In some aspects, the present disclosure relates to pharmaceutical compositions comprises of composite particles containing niclosamide, human lysozyme, and stabilizing excipients (referred to as NIC-hLYS herein) capable of being delivered to the upper and lower airways in the treatment of diseases of the respiratory tract including viral infections, bacterial infections, inflammatory lung diseases (asthma, COPD, bronchiectasis), and lung cancer. The composite particles are engineered in such a way that the resulting composition may be delivered in powder form using a dry powder inhaler (DPI) to the lower airways, or may be reconstituted in an aqueous, physiologically-compatible medium for delivery to the lower airways via nebulizer or delivery to the upper airways via an actuated nasal spray. The ability to deliver the pharmaceutical compositions using a range of delivery systems without the need for changes to the powder components and ratios or processing methods makes the composition broadly applicable to a range of patient populations, and includes those who are ambulatory or in an out-patient setting, patients with reduced lung function or those who may require mechanical ventilation, and pediatric or geriatric who may exhibit reduced inspiratory capacity.

Such a feature is particularly valuable for the treatment of COVID-19, in which a considerable challenge for drug product development is the variable presentation of illness. Patients may serve as asymptomatic carriers of the virus or develop severe pneumonia and acute respiratory disease, which can result in the requirement for mechanical ventilation (Lai et al., 2020). In the case of ventilated patients, aerosol drug delivery is typically performed using a nebulizer. However, for treatment of asymptomatic carriers or in developing regions with reduced access to clean water sources, nebulizer-based therapy may present an undue burden and reduce therapy compliance. For these populations, a DPI or nasal spray, or a combination of both, would be the preferred option based upon the rapid administration time and ease of use and could also potentially be utilized as a prophylactic therapy in high risk populations such as healthcare workers and first responders. Rather than developing three separate compositions for to meet the needs of these diverse groups, the utilization of the same composition for multiple delivery systems would enable rapid scale-up of production and would improve global access and adoption of the product during the pandemic.

The high potency of NIC when administered directly to the site of disease may require the utilization of diluents for dose-filling and dose delivery purposes. DPI delivery systems typically utilize large lactose carrier particles to dilute potent drugs. Likewise, preparation of NIC as a suspension for nasal or oral inhalation requires the use of stabilizing and viscosity enhancing agents to prevent aggregation and settling of the active drug and ensure the correct dose is administered. However, only a limited number of excipients are approved by regulatory agencies for the nasal route, and even fewer for the pulmonary route. An endogenous protein called human lysozyme was found to generate a stable suspension of micron-sized NIC particles which exhibited an acceptable size distribution for oral inhalation (median diameter <5 μm). Furthermore, it was determined that spray drying a suspension incorporating a specific ratio of NIC to lysozyme with additional excipients to stabilize the protein results in a dry powder that exhibits a suitable size distribution for oral inhalation (median diameter <5 μm) which is maintained upon reconstitution of the powder in an aqueous medium. Additionally, achieving complete dispersion of the cohesive, micronized NIC particles upon reconstitution of the spray dried composition does not require sonication or vortexing of the suspension, which may result in damage and immunogenic aggregation of the lysozyme component. Instead, a particle size distribution appropriate for nasal spray administration or nebulizer-based administration is achieved by simply adding the reconstituting media and gently inverting the suspension. This enables the reconstitution procedure to be performed at the time of care.

Also provided herein are methods of preparing and using these compositions. Details of these compositions are provided in more detail below.

### I. Pharmaceutical Compositions

In some aspects, the present disclosure provides pharmaceutical compositions containing niclosamide and a protein that may be formulated for administration to the lungs.
A. Niclosamide

The pharmaceutical compositions described herein comprise niclosamide as an active agent. The pharmaceutical compositions described herein contain niclosamide in an amount between about 0.1% to about 20% w/w, between about 0.1% to about 10% w/w, between about 0.2% to about 5% w/w, or between about 0.5% to about 1% w/w of the total composition. In some embodiments, the amount of the niclosamide is from about 0.1%, 0.2%, 0.3%, 0.4%, 0.5%, 0.6%, 0.65%, 0.7%, 0.75%, 0.8%, 0.9%, 1%, 2%, 3%, 4%, 5%, 6%, 8%, 30 10%, 15%, to about 20% w/w or any range derivable therein.

In some aspects, a wide variety of different forms of niclosamide may be used. Niclosamide is an active agent with a chemical name of 5-Chloro-N-(2-chloro-4-nitrophenyl)salicylamide. The niclosamide used herein may be either anhydrous or may be a hydrate of niclosamide such as monohydrate of niclosamide. Furthermore, the niclosamide may be a salt such as an ethanolamine or piperazine salt. Additionally, co-crystal of niclosamide may be used in the pharmaceutical compositions may include co-crystals of niclosamide with 2-aminothiazole, benzamide, isoniazid, acetamide, caffeine, urea, p-aminobenzoic acid, theophylline, nicotinamide, or isonicotinamide (Sanphui et al., 2012; Luedeker et al., 2016). Alternative, it is also contemplated that known derivatives such as those described by Mook et al., 2015, which is incorporated herein by reference may also be used in the formulations. Additionally, niclosamide is light sensitive and should be stored in the dark to protect the composition from light.

### 1. Inhalation

In some embodiments, the present disclosure relates to respirable particles must be in the aerodynamic size range, such as mean median aerodynamic diameter of around 2 to 10 microns or 4 to 8 microns in aerodynamic diameter. In some embodiments, the present disclosure provides methods for the administration of the inhalable niclosamide composition provided herein using a device. Administration may be, but is not limited, to inhalation of niclosamide using an inhaler. In some embodiments, an inhaler is a simple passive dry powder inhaler (DPI), such as a Plastiape RSO1 monodose DPI. In a simple dry powder inhaler, dry powder is stored in a capsule or reservoir and is delivered to the lungs by inhalation without the use of propellants.

In some embodiments, an inhaler is a single use, disposable inhaler such as a single-dose DPI, such as a DoseOne™, Spinhaler, Rotohaler®, Aerolizer®, or Handihaler. These dry powder inhaler may be a passive DPI. In some embodiments, an inhaler is a multidose DPI, such as a Plastiape RS02, Turbuhaler®, Twisthaler™, Diskhaler®, Diskus®, or Ellipta™. In some embodiments, the inhaler is Twincer®, Orbital®, TwinCaps®, Powdair, Cipla Rotahaler, DP Haler, Revolizer, Multi-haler, Twister, Starhaler, or Flexhaler®. In some embodiments, an inhaler is a plurimonodose DPI for the concurrent delivery of single doses of multiple medications, such as a Plastiape RS04 plurimonodose DPI. Dry powder inhalers have medication stored in an internal reservoir, and medication is delivered by inhalation with or without the use of propellants. Dry powder inhalers may require an inspiratory flow rate greater than 30 L/min for effective delivery, such as between about 30-120 L/min.

In some embodiments, the inhalable niclosamide is delivered as a propellant formulation, such as HFA propellants.

In some embodiments, the inhaler may be a metered dose inhaler. Metered dose inhalers deliver a defined amount of medication to the lungs in a short burst of aerosolized medicine aided by the use of propellants. Metered dose inhalers comprise three major parts: a canister, a metering valve, and an actuator. The medication formulation, including propellants and any required excipients, are stored in the canister. The metering valve allows a defined quantity of the medication formulation to be dispensed. The actuator of the metered dose inhaler, or mouthpiece, contains the mating discharge nozzle and typically includes a dust cap to prevent contamination.

In some embodiments, an inhaler is a nebulizer or a soft-mist inhaler such as those described in WO 1991/14468 and WO 1997/12687, which are incorporated herein by reference. A nebulizer is used to deliver medication in the form of an aerosolized mist inhaled into the lungs. The medication formulation be aerosolized by compressed gas, or by ultrasonic waves. A jet nebulizer is connected to a compressor. The compressor emits compressed gas through a liquid medication formulation at a high velocity, causing the medication formulation to aerosolize. Aerosolized medication is then inhaled by the patient. An ultrasonic wave nebulizer generates a high frequency ultrasonic wave, causing the vibration of an internal element in contact with a liquid reservoir of the medication formulation, which causes the medication formulation to aerosolize. Aerosolized medication is then inhaled by the patient. In some embodiments, the single use, disposable nebulizer may be used herein. A nebulizer may utilize a flow rate of between about 3-12 L/min, such as about 6 L/min. In some embodiments, the nebulizer is a dry powder nebulizer.

In some embodiments, the composition may be administered on a routine schedule. As used herein, a routine schedule refers to a predetermined designated period of time. The routine schedule may encompass periods of time which are identical, or which differ in length, as long as the schedule is predetermined. For instance, the routine schedule may involve administration four times a day, three times a day, twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In some embodiments, niclosamide is administered once per day. In preferred embodiments, niclosamide is administered less than once per day, such as every other day, every third day, or once per week. In some embodiments, a complete dose of niclosamide is between 0.05-30 mg, such as 0.1-10, 0.25-5, 0.3-5, or 0.5-5 mg.

In some embodiments, the amount of niclosamide of the nebulizer or inhaler may be provided in a unit dosage form, such as in a capsule, blister or a cartridge, wherein the unit dose comprises at least 0.05 mg of niclosamide, such as at least 0.075 mg or 0.100 mg of niclosamide per dose. In particular aspects, the unit dosage form does not comprise the administration or addition of any excipient and is merely used to hold the powder for inhalation (i.e., the capsule, blister, or cartridge is not administered). In some embodiments, the entire amount of the powder load may be administered in a high emitted dose, such as at least 1 mg, preferably at least 10 mg, even more preferably 50 mg. In some embodiments, administration of the powder load results in a high fine particle dose into the deep lung such as greater than 1 mg. Preferably, the fine particle dose into the deep lung is at least 5 mg, even more preferably at least 10 mg. In some embodiments, the dose may further comprise using a dose from a reservoir or non-unit dose form and the relevant dose is metered out from the device such as a nasal spray or turbuhaler.
3. Uses of Compositions

Several clinical indications would benefit from administration of niclosamide compositions with enhanced bioavailability. These indications include the infections of a microorganism such as bacteria, a virus, a parasite, or a worm. In particular, the compositions may be used to treat a viral infection. Some non-limiting examples of viral infections which may be treated with the composition described herein include COVID-10, MERS, SARS, influenza, respiratory syncytial (RSV), Zika, Lassa, Ebola, HIV including HIV with complications such as TB, and adenovirus. In other embodiments, the pharmaceutical compositions may be used to treat schistosomiasis and related pulmonary complications. Additionally, these pharmaceutical compositions may be used to treat vancomycin resistant enterococci, Pseudomonas aeruginosa, Acinetobacter baumannii, klebsiella pneumoniae, C. difficile, or MRSA. Furthermore, the pharmaceutical composition may be used to treat or control diabetes. With regards to viral infections, some viruses such as SARS-CoV can enter cells and replicate where ACE2+tissues are present, which includes areas such as the kidneys, lungs and small intestine (Hoffmann et al., 2020). Other such clinical indications include several cancers, in particular castration-resistant prostate cancer, glioblastoma, esophageal cancer, or lung cancer. In particular, these compositions may be used in the treatment of cancer sassociated with the STATS pathway appear to be particular active. The pharmaceutical composition may be administered in combination with a PD1 inhibitor.

In some embodiments, the pharmaceutical composition may be used to treat one or more diseases or disorders in combination with one or more additional active agents. In particular, the pharmaceutical composition may be used in conjunction with another antimicrobial agent or active agent which reduces one or more symptoms of the microbial infection. Some non-limiting examples of additional therapeutic agents may include chloroquine, hydroxychloroquine, thalidomide, plasminogen, colistin, polymyxin B, or clofazimine. In other compositions, the pharmaceutical composition may be used in conjunction with one or more anti-cancer agents such as a chemotherapeutic agent, radiotherapy, surgery, or immunotherapy. Some non-limiting examples of additional therapeutic agents may include abiraterone such as abiraterone acetate, enzalutamide, bicalutamide, erlotinib, a PD-Ll antibody, a platinum drug, or a taxane based drug.

### B. Protein

The pharmaceutical compositions described herein comprise protein such as a protein which is positively charged at physiological pH. This particular protein may be an endogenous protein. The protein may function to modulate the immune system in vivo or show one or more therapeutic effects against an indication. An immunomodulating protein is one that causes a change in one or more markers of the immune system such as modulating the expression of an interleukin. The pharmaceutical compositions described herein contain a protein in an amount between about 20% to about 95% w/w, between about 40% to about 95% w/w, between about 50% to about 90% w/w, or between about 55% to about 85% w/w of the total composition. In some embodiments, the amount of the protein is from about 20%, 25%, 30%, 35%, 40%, 40%, 45%, 50%, 55%, 60%, 62.5%, 65%, 67.5%, 70%, 72.5%, 75%, 77.5%, 80%, 85%, 90%, to about 95% w/w or any range derivable therein.

In some aspects, a wide variety of different forms of proteins may be contemplated to be used in the formulations described herein. In particular, these proteins may be ones that have been humanized or a human protein. The amino acid sequence of the protein may have also been modified in such a way that it reduces the degradation of the protein or immunogenicity. These modifications may alter the protein from being degraded during formulation, during storage, or in vivo.

In some embodiments, the protein used herein is lysozyme, such as human lysozyme. Lysozyme is an enzymatic protein that functions as a part of the immune system by acting as a glycoside hydrolase. The enzyme catalyzes the hydrolysis of 1,443-linkages between N-acetylmuramic acid and N-acetyl-D-glucosamine thus is useful in the lysis of bacteria.

### C. Excipients

In some aspects, the present disclosure comprises one or more excipients formulated into pharmaceutical compositions. An “excipient” refers to pharmaceutically acceptable carriers that are relatively inert substances used to facilitate administration or delivery of an API into a subject or used to facilitate processing of an API into drug 15 formulations that can be used pharmaceutically for delivery to the site of action in a subject.

Furthermore, these compound may be used as diluents in order to obtain a dosage that can be readily measured or administered to a patient. Non-limiting examples of excipients include stabilizing agents, surfactants, surface modifiers, solubility enhancers, buffers, encapsulating agents, antioxidants, preservatives, nonionic wetting or clarifying agents, viscosity increasing agents, and absorption-enhancing agents.

In some aspects, the amount of the excipient in the pharmaceutical composition is from about 0.00001% to about 70% w/w, from about 0.001% to about 40% w/w, from about 0.01% to about 30% w/w, or from about 0.1% to about 20% w/w. The amount of the excipient in the pharmaceutical composition comprises from about 0.001%, 0.005%, 0.01%, 0.05%, 0.1%, 0.125%, 0.15%, 0.2%, to about 0.25% w/w, or any range derivable therein, of the total pharmaceutical composition. In one embodiment, the amount of the excipient in the pharmaceutical composition is at 0.05% to 0.25% w/w of the total weight of the pharmaceutical composition. Alternatively, the amount of the excipient in the pharmaceutical composition comprises from about 1%, 5%, 10%, 15%, 20%, 25%, 30%, 35%, 40%, 45%, 50%, 55%, 60%, 70%, to about 80% w/w, or any range derivable therein, of the total pharmaceutical composition. In one embodiment, the amount of the excipient in the pharmaceutical composition is at 20% to 40% w/w of the total weight of the pharmaceutical composition.

In some aspects, the present disclosure may further comprise one or more excipient such as a saccharide or a surfactant. Some composition may further comprise a mixture of two or more excipients including two or more surfactants.

### 1. Saccharides

In some aspects, the present disclosure comprises one or more excipients formulated into pharmaceutical compositions. In some embodiments, the excipients used herein are water soluble excipients. These saccharides may be used to act as a lyoprotectant that protects the protein from destabilization during the drying process. These water-soluble excipients include carbohydrates or saccharides such as disaccharides such as sucrose, trehalose, or lactose, a trisaccharide such as fructose, glucose, galactose comprising raffinose, polysaccharides such as starches or cellulose, or a sugar alcohol such as xylitol, sorbitol, or mannitol. In some embodiments, these excipients are solid at room temperature. Some non-limiting examples of sugar alcohols include erythritol, threitol, arabitol, xylitol, ribitol, mannitol, sorbitol, galactitol, fucitol, iditol, inositol, volemitol, isomalt, maltitol, lactitol, maltotritol, maltotetraitol, or a polyglycitol. In other aspects, larger molecules like amino acids, peptides and proteins are incorporated to facilitate inhalation delivery, including leucin, trileucine, histidine and others.

### 2. Surfactants

In some embodiments, the present disclosure comprises one or more surfactants. The surfactant may be a fatty acid, a triglyceride, an ester of a fatty acid, or mixtures thereof. In the formulation, the surfactant may be used to help separate the protein at the air-liquid interface and help to suspend the niclosamide in the solution. The term lipid includes fatty acids which are a group of aliphatic saturated or unsaturated carboxylic acids. The chains are usually unbranched and have 6 to 30, preferably 8 to 22, and in particular 8 to 18, carbon atoms. Some non-limiting examples of saturated fatty acids include caproic acid, enanthic acid, caprylic acid, pelargonic acid, capric acid, undecanoic acid, lauric acid, tridecanoic acid, myristic acid, pentadecanoic acid, palmitic acid, margaric acid, stearic acid, nonadecanoic acid, arachidic acid, behenic acid, lignoceric acid, cerotic acid and melissic acid. Additionally, the term includes unsaturated fatty acids may be unsaturated one or more times, in particular unsaturated once, twice, three times, four times, five times or six times. Some non-limiting examples of singly unsaturated fatty acids include palmitoleic acid, oleic acid and erucic acid, of doubly unsaturated fatty acids include sorbic acid and linoleic acid, of triply unsaturated fatty acids include linolenic acid and eleostearic acid, of quadruply unsaturated fatty acids include arachidonic acid, of quintuply unsaturated fatty acids include clupanodonic acid, and of sextuply unsaturated fatty acids include docosahexaenoic acid.

As used herein, the term surfactant refers to a compound which exhibits amphiphilic character and reduces the surface tension of a solvent, particularly water. Surfactants can generally be classified into four categories: cationic, anionic, zwitterionic, or non-ionic. While it is contemplated that any of these surfactants may be used in the present compositions, non-ionic surfactant shows particular promise. Cationic surfactants include, but are not limited to, amines with long alkyl chains and are protonated at a physiologically relevant pH or permanently charged quaternary ammonium salts such as cetrimonium bromide, cetylpyridinium chloride, benzalkonium chloride, benzethonium chloride, dimethyldioctadecylammonium chloride, or dioctadecyldimethylammonium bromide. Some non-limiting examples of anionic surfactants include sulfate, sulfonate, or phosphate esters such as docusate, perfluorooctanesulfonate, perfluorobutanesulfonate, alkyl-aryl ether phosphates, or alkyl ether phosphate or carboxylate esters including alipahtic carboxylates such as fatty acids and derivatives thereof. Other examples of zwitterionic surfactants including phospholipids such as phosphotidylserine, phosphotidylcholine, phosphotidylethanolamine, or sphingomyelins, sultaines such as CHAPS and cocamidopropyl hydroxysultaine, or betaine such as cocamidopropyl betaine. Finally, some non-limiting examples of nonionic surfactants include PEG alkyl ethers, polypropylene glycol ethers, glucoside alkyl ethers, PEG alkylaryl ethers such as Triton® and nonoxynol, simple alkyl esters of glycerol such as glycerol laurate, polysorbates such as Tween, Sorbitan alkyl esters such as Span, or poloxamer and other block copolymers of polyethylene glycol and polypropylene glycol. In some embodiments, the surfactants used in the present pharmaceutical compositions contain one or more polyethylene glycol or polypropylene glycol polymer such as Tween, Capryol, Labrafil, or Labrasol.

In some aspects, the present disclosure provides a surfactant which includes a PEG polymer with a molecular weight from about 100 to about 4000 daltons, from about 100 to about 1000 daltons, from about 100 to about 500 daltons, or from about 100, 200, 300, 400, 500, 600, 700, 800, 900, 1000, 1250, 1500, 1750, 2000, 2500, 3000, 3500, or about 4000 daltons. In some embodiments, the PEG polymer further comprises a hydrophobic group such as a vitamin or fatty acid or is a PEGylated version of sorbate such as Tween.

### 3. Buffers

In some embodiments, the compositions may further comprise a buffer. A buffer is a mixture of molecules within the solution which help to maintain a constant pH. Common buffers that may be used in phosphate, bicarbonate, HEPES, histidine, or Tris. In some aspects, the compositions herein may be reformulated using a buffer such as histidine. The buffer helps to stabilize the protein so that the protein does not crystalize during the drying or freezing processes as well as keep the protein intact during the process and reconstitution. The pharmaceutical compositions may comprises from about 0.25 mg/mL of the buffer to about 5 mg/mL of the buffer, such as from about 0.5 mg/mL to about 2.5 mg/mL of the buffer. The pharmaceutical composition may comprise from 0.1 mg/mL, 0.2 mg/mL, 0.25 mg/mL, 0.4 mg/mL, 0.5 mg/mL, 0.75 mg/mL, 1 mg/mL, 1.2 mg/mL, 1.4 mg/mL, 1.5 mg/mL, 1.6 mg/mL, 1.8 mg/mL, 2 mg/mL, 2.2 mg/mL, 2.4 mg/mL, 2.5 mg/mL, 2.6 mg/mL, 2.8 mg/mL, to about 3 mg/mL of the buffer, or any range derivable therein.

### II. Manufacturing Methods

A. Micronization and Jet Milling

As used in this application, the pharmaceutical composition may comprise using a component that has been “micronized” which refers to a substance, such as an active agent, that has been broken down into very fine particles, typically less than 10 μm, preferably between 0.5 and 5 μm, more preferably between 1 and 3 μm. A substance may be micronized by milling, grinding, or crushing Milling may be performed by any method known in the art, such as by air jet mill, ball mill, wet mill, high pressure homogenization, or cryogenic mill.

In some aspects, “air jet mill” which is a device or method for reducing particle size by using a jet of compressed gas to impact particles into one another or the walls of the mill, thereby pulverizing the particles. An air jet mill may be used to micronize particles. Air jet mills are commercially available, such as the Aljet Model 00 Jet-O-Mizer™ (Fluid Energy, Telford, PA).

Alternatively, the pharmaceutical composition may be subjected to a “ball mill” which is a device or method for reducing particle size by adding the particle of interest and a grinding medium to the interior of a cylinder and rotating the cylinder. The particles of interest are broken down as the grinding medium rises and falls along the exterior of the cylinder as it rotates.

Furthermore, the pharmaceutical composition may be subjected to a “wet mill” or “media mill” which is a device or method for reducing particle size by adding the particle of interest to device with an agitator, containing a media comprising a liquid and a grinding medium. With the addition of the particle of interest, as the agitator rotates, the energy it disperses causes the grinding medium and particles of interest to come into contact and break down the particles of interest.

In other embodiments, the pharmaceutical composition may be subjected to a “high pressure homogenization” which is a device or a method of reducing particle size by adding the particle of interest to a device which combines both pressure and mechanical forces to break down the particle of interest. Mechanical forces used in high pressure homogenization may include impact, shear, and cavitation, among others.

As used herein in the specification and the claims, the term “cryogenic mill” refers to a device or method for reducing particle size by first chilling a particle of interest with dry ice, liquid nitrogen, or other cryogenic liquid, and subsequently milling the particle of interest to reduce the size.

### B. Spray Drying

Thus, the final formulations may be prepared using a spray drying technique. Spray drying is a process that converts a liquid feed to a dried particulate form. The process may further comprise a secondary drying process, such as fluidized bed drying or vacuum drying, may be used to reduce residual solvents to pharmaceutically acceptable levels. Typically, spray-drying involves contacting a highly dispersed liquid suspension or solution with a sufficient volume of hot air or other gas to produce evaporation and drying of the liquid droplets. In a standard procedure, the composition is sprayed into a current of warm filtered air or gas that evaporates the solvent and conveys the dried product to a collector. The spent air is then exhausted with the solvent, or alternatively the spent air is sent to a condenser to capture and potentially recycle the solvent. The spray is emitted through a nozzle such as a pressure nozzle, a two-fluid electrosonic nozzle, a two-fluid nozzle, or a rotary atomizer. Commercially available types of apparatus may be used to conduct the spray-drying such as those manufactured by Buchi Ltd. and Niro or described in US 2004/0105820 and US 2003/0144257.

Spray-drying typically employs a solids loading of material from about 0.25% to about 30% such as about 1% solids loading. If the solids loading is too low, then the composition may be unable to be formulated commercially or result in a product that is too dilute to be useful. On the other hand, the upper limit of solids loading is governed by the viscosity of the resulting solution and the solubility of the components in the solution. This material may be fed from the spray dryer at a feed flow rate from about 0.01 mL/min to about 100 mL/min. from about 0.05 mL/min to about 50 mL/min, or from about 0.1 mL/min to about 40 mL/min. In some embodiments, the feed flow rate may be from about 0.01 mL/min, 0.05 mL/min, 0.1 mL/min, 0.2 mL/min, 0.5 mL/min, 1 mL/min, 2.5 mL/min, 5 mL/min, 7.5 mL/min, 10 mL/min, 12.5 mL/min, 15 mL/min, 20 mL/min, 25 mL/min, 30 mL/min, 35 mL/min, 40 mL/min, 45 mL/min, 50 mL/min, 75 mL/min, to about 100 mL/min, or any range derivable therein.

Techniques and methods for spray-drying may be found in Perry's Chemical Engineering Handbook, 6th Ed., R. H. Perry, D. W. Green & J. O. Maloney, eds.), McGraw-Hill book co. (1984); and Marshall “Atomization and Spray-Drying” 50, Chem. Eng. Prog. Monogr. Series 2 (1954). In general, the spray-drying is conducted with an inlet temperature of from about 40° C. to about 200° C., for example, from about 80° C. to about 180° C., or from about 100° C. to about 140° C. The inlet temperature may from about 35° C., 40° C., 50° C., 60° C., 70° C., 80° C, 90° C., 100° C., 110° C., 120° C., 130° C., 140° C., 150° C., 160° C., 170° C., 180° C., to about 200° C., or any range derivable therein.

### III. Definitions

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “drug”, “pharmaceutical”, “active agent”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

The terms “compositions,” “pharmaceutical compositions,” “formulations,” “pharmaceutical formulations,” “preparations”, and “pharmaceutical preparations” are used synonymously and interchangeably herein.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis (3-hydroxy-2-ene-1-carboxylic acid), 4-methylbicyclol2.2.2loct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “derivative thereof” refers to any chemically modified polysaccharide, wherein at least one of the monomeric saccharide units is modified by substitution of atoms or molecular groups or bonds. In one embodiment, a derivative thereof is a salt thereof. Salts are, for example, salts with suitable mineral acids, such as hydrohalic acids, sulfuric acid or phosphoric acid, for example hydrochlorides, hydrobromides, sulfates, hydrogen sulfates or phosphates, salts with suitable carboxylic acids, such as optionally hydroxylated lower alkanoic acids, for example acetic acid, glycolic acid, propionic acid, lactic acid or pivalic acid, optionally hydroxylated and/or oxo-substituted lower alkanedicarboxylic acids, for example oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example methanesulfonates, benzenesulfonates, p-toluenesulfonates or N-cyclohexylsulfamates (cyclamates).

The term “dissolution” as used herein refers to a process by which a solid substance, here the active ingredients, is dispersed in molecular form in a medium. The dissolution rate of the active ingredients of the pharmaceutical dose of the invention is defined by the amount of drug substance that goes in solution per unit time under standardized conditions of liquid/solid interface, temperature and solvent composition.

As used herein, the term “aerosols” refers to dispersions in air of solid or liquid particles, of fine enough particle size and consequent low settling velocities to have relative airborne stability (See Knight, V., Viral and Mycoplasmal Infections of the Respiratory Tract. 1973, Lea and Febiger, Phila. Pa., pp. 2).

As used herein, the term “physiological pH” refers to a solution with is at its normal pH in the average human. In most situation, the solution has a pH of approximately 7.4.

As used herein, “inhalation” or “pulmonary inhalation” is used to refer to administration of pharmaceutical preparations by inhalation so that they reach the lungs and in particular embodiments the alveolar regions of the lung. Typically inhalation is through the mouth, but in alternative embodiments in can entail inhalation through the nose.

As used herein, “dry powder” refers to a fine particulate composition that is not suspended or dissolved in an aqueous liquid.

A “simple dry powder inhaler” refers a device for the delivery of medication to the respiratory tract, in which the medication is delivered as a dry powder in a single-use, single-dose manner In particular aspects, a simple dry powder inhaler has fewer than 10 working parts. In some aspects, the simple dry powder inhaler is a passive inhaler such that the dispersion energy is provided by the patient's inhalation force rather than through the application of an external energy source.

A “median particle diameter” refers to the geometric diameter as measured by laser diffraction or image analysis. In some aspects, at least either 50% or 80% of the particles by volume are in the median particle diameter range.

A “Mass Median Aerodynamic Diameter (MMAD)” refers to the aerodynamic diameter (different than the geometric diameter) and is measured by laser diffraction.

The term “amorphous” refers to a noncrystalline solid wherein the molecules are not organized in a definite lattice pattern. Alternatively, the term “crystalline” refers to a solid wherein the molecules in the solid have a definite lattice pattern. The crystallinity of the active agent in the composition is measured by powder x-ray diffraction.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significant such as “significantly”) is not meant to imply statistical differences between two values but only to imply importance or the scope of difference of the parameter.

Throughout this application, the term “about” is used to indicate that a value includes the inherent variation of error for the device, the method being employed to determine the value, or the variation that exists among the study subjects or experimental studies. Unless another definition is applicable, the term “about” refers to ±5% of the indicated value.

As used herein, the term “substantially free of” or “substantially free” in terms of a specified component, is used herein to mean that none of the specified component has been purposefully formulated into a composition and/or is present only as a contaminant or in trace amounts. The total amount of all containments, by-products, and other material is present in that composition in an amount less than 2%. The term “essentially free of” or “essentially free” is used to represent that the composition contains less than 1% of the specific component. The term “entirely free of” or “entirely free” contains less than 0.1% of the specific component.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements and parameters.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### IV. Examples

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1—Optimization of Pharmaceutical Composition Components

Niclosamide was micronized using an air jet mill (Fluid Energy) to achieve an X90 diameter of 4.2 μm. The micronized powder was used in the preparation of all compositions.

Feed stock suspensions for spray drying were generated by mixing micronized niclosamide, sucrose, and lyophilized human lysozyme in a mortar and pestle using a process of geometric dilution. Polysorbate 80 (prepared as an aqueous solution of 10 mg/mL polysorbate 80) was added to wet the powder. 5 mL of a 1.74 mg/mL histidine aqueous solution was added in 1 mL increments to the mortar with continuous mixing. The suspension was poured into separate container. 5 mL of charcoal filtered water was added to the mortar and mixed in 1 mL increments. This was then added to the suspension container. The mortar was then washed with water in 5 mL increments until a 50 mL final volume was reached. The suspension was gently inverted to mix, and then spray dried using a BUCHI B290 spray dryer with dehumidifier attachment using air as the atomization gas (22.9 L/min), inlet temperature set to 130° C., and feed flow rate set to 1 mL/min

The PSD of the powder was measured using a Sympatec laser diffractor unit with a RODOS powder disperser attachment. The dispersion pressure was set at 3 bar and the rotary feed table set at 20% of the maximum rotation speed.

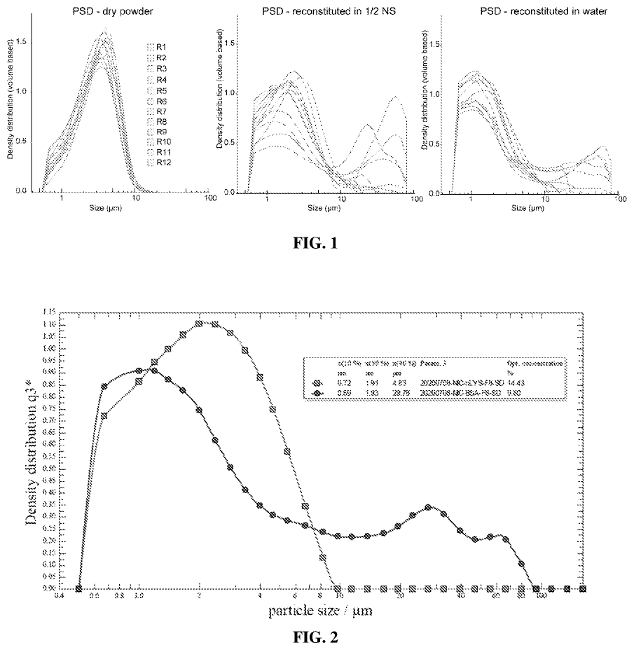

The excipient ratios of the example formulations and the resulting powder PSD and suspension PSD are presented in Table 1 and graphically in FIG. 1. The formulations that are bolded in the table are those deemed to be particularly promising based upon the resulting powder and suspension PSD. Formulation 8 (F8) was selected for further characterization.

TABLE 1
Formulations for production of inhalable niclosamide particles and resulted particle size distributions

| | | | | | X90 | Reconstituted in ½ NS | | | Reconstituted in DI water | | | Decrease |
|---|---|---|---|---|---|---|---|---|---|---|---|---|
| | Micronized | Human | | | dia- | X50 | X90 | X99 | X50 | X90 | X99 | in |
| | niclos- | Lyso- | | Tween | meter- | dia- | dia- | dia- | dia- | dia- | dia- | monomer |
| | amide | zyme | Sucrose | 80 | dry powder | meter | meter | meter | meter | meter | meter | vs control |
| Formulation | (% w/w) | (% w/w) | (% w/w) | (% w/w) | (μm) | (μm) | (μm) | (μm) | (μm) | (μm) | (μm) | (%) |
| 1 | 0.30 | 60.00 | 39.65 | 0.050 | 5.63 | 5.16 | 42.13 | 83.65 | 1.41 | 8.66 | 18.72 | 0.00 |
| 2 | 1.00 | 60.00 | 38.95 | 0.050 | 5.80 | 2.44 | 6.49 | 15.48 | 1.87 | 47.47 | 107.46 | 0.00 |
| 3 | 0.30 | 79.65 | 20.00 | 0.050 | 6.07 | 2.23 | 36.32 | 73.41 | 2.18 | 41.64 | 76.01 | 0.00 |
| 4 | 1.00 | 78.95 | 20.00 | 0.050 | 6.66 | 1.91 | 4.42 | 7.18 | 1.39 | 4.08 | 12.06 | 0.00 |
| 5 | 0.30 | 60.00 | 39.50 | 0.200 | 6.32 | 19.73 | 72.22 | 104.27 | 1.93 | 30.48 | 66.36 | 0.00 |
| 6 | 0.30 | 79.50 | 20.00 | 0.200 | 6.29 | 1.58 | 5.05 | 11.84 | 2.08 | 39.65 | 72.61 | 0.00 |
| 7 | 1.00 | 60.00 | 38.80 | 0.200 | 5.46 | 1.84 | 5.57 | 23.03 | 1.69 | 6.06 | 39.35 | 0.00 |
| 8 | 1.00 | 78.80 | 20.00 | 0.200 | 6.11 | 1.77 | 5.10 | 12.22 | 1.49 | 4.11 | 8.35 | 0.00 |
| 9 | 1.00 | 78.88 | 20.00 | 0.125 | 5.62 | 1.64 | 5.78 | 21.74 | 1.34 | 3.87 | 11.23 | 0.00 |
| 10 | 0.30 | 69.79 | 29.79 | 0.125 | 5.95 | 2.98 | 54.57 | 81.88 | 1.81 | 50.22 | 81.49 | 0.09 |
| 11 | 0.65 | 60.00 | 39.22 | 0.125 | 6.06 | 2.10 | 14.64 | 38.86 | 1.41 | 4.10 | 11.58 | 0.00 |
| 12 | 0.65 | 69.65 | 29.65 | 0.050 | 6.56 | 1.70 | 5.11 | 14.66 | 1.42 | 5.36 | 22.13 | 0.00 |

*All formulations prepared in 0.174 mg/mL histidine

Formulation F8 was further characterized. Upon drying, the NIC content of the formulation was found to typically range from 0.67% to 0.74% w/w. Using Karl Fisher titration, the water content of the powder was determined to be 8.78%. The glass transition temperature of the powder was found to 79.4° C., which makes it well suited for storage in ambient conditions. Lastly, the osmolality of the various concentrations of the powder suspended in 0.45% sodium chloride was assessed with a freezing-point depression osmometer (Precision Systems Model 5004). After calibration of the instrument, 50 μL of sample suspension was added to sample chamber. The resulting osmolality measurements are presented in Table 2.

TABLE 2
Osmolality of various concentrations
of niclosamide-lysozyme suspensions

| Concentration | Osmolality |
|---|---|
| (mg/mL) | (mOsmol/kg) |
| 10 | 156 ± 3 |
| 25 | 179 ± 4 |
| 50 | 208 ± 1 |

### Example 2—Pharmaceutical Characteristics of the Niclosamide Protein Compositions

### A. Proposed Protein Interactions

To evaluate the utility of human lysozyme in generating a stable suspension of micronized niclosamide, an additional formulation utilizing bovine serum albumin was developed, which contained the same weight-based ratios as the optimized F8 formulation. Lysozyme is positively charged at physiological pH, while albumin is negatively charged. Thus, the experiment served to examine the influence of charge on the stabilizing activity of the protein. Laser diffraction measurement of the resulting niclosamide suspensions prepared from the two formulations are presented in FIG. 2. NIC-BSA exhibits a broad particle size distribution unsuitable for nebulization, while NIC-hLYS exhibits a narrower particle size distribution which would be appropriate for aerosolization using a vibrating mesh nebulizer.

Protein aggregation may result in the potentially loss of therapeutic activity or an immunogenic response (Wang et al., 2012; Ratanji et al., 2014; Moussa et al., 2016). It has been previously demonstrated that lysozyme is robust to process-induced aggregation in typical particle engineering techniques, (Brunaugh et al., 2019) which provided part of the rationale for its selection as a therapeutically active carrier for the aerosol delivery of NIC particles. Using size exclusion chromatography (SEC), the formation of higher molecular weight hLYS aggregates before and after spray drying was investigated. No further increases in higher molecular weight aggregates were noted after spray drying (FIG. 8B) or after nebulization at varying reconstituted concentrations (FIGS. 8C-8F) (Table 2). Interestingly, a decrease in the percentage of solubilized aggregates was noted in the spray dried hLYS formulations compared to the initial unprocessed hLYS product, which may be explained by the shift in secondary structure from towards a higher percentage of parallel b-sheet structure upon spray drying, whereas the unprocessed hLYS had a higher percentage of anti-parallel b-sheet structure (Table 3). The glass transition temperature (Tg) measured for the NIC-hLYS powder was 79.4° C., which makes it suitable for storage in ambient conditions without risk of further protein degradation. The water content of the spray dried powder was determined to be 8.8% based upon Karl Fisher coulometric titration, which is similar to literature reported values for the water content of lysozyme.(Elkordy et al., 2002)

TABLE 3
Aggregation of hLYS before and after spray drying and nebulization

| Sample | HMW (%) | Monomer (%) | Fragment (%) |
|---|---|---|---|
| hLYS unprocessed | 17.2 | 82.1 | 0.7 |
| NIC-hLYS-SD | 13.7 | 84.3 | 2.1 |
| 25 mg/mL nebulized | 14.1 | 84.2 | 1.7 |
| 50 mg/mL nebulized | 13.4 | 84.7 | 1.9 |
| 75 mg/mL nebulized | 12.5 | 85.8 | 1.6 |
| 100 mg/mL nebulized | 14.0 | 84.4 | 1.7 |

| | Anti-parallel | Parallel | | | |
|---|---|---|---|---|---|
| | β-sheet (%) | β-sheet (%) | α-helix (%) | Turns (%) | |
| Sample | (1618-1625 cm⁻¹) | (1633-1637 cm⁻¹) | (1653 cm⁻¹) | (1678-1680 cm⁻¹) | Adj R² |
| hLYS | 21.7 | 0.7 | 57.6 | 20.2 | 0.99 |
| unprocessed | | | | | |
| hLYS-SD | 10.4 | 12.1 | 59.5 | 18.1 | 0.99 |
| NIC-hLYS-SD | 12.6 | 9.7 | 58.5 | 19.2 | 0.99 |

B. Pharmaceutical Composition Formulations with 3 Different Types of Aerosol Delivery Methods

Targeted delivery of antivirals to the respiratory tract carries substantive benefits for the treatment of COVID-19, particularly for compounds with limited oral bioavailability. However, the wide range of symptoms and disease severity associated with COVID-19 makes development of a broadly applicable therapy difficult. The clinical applicability of the NIC-hLYS formulation was assessed by determining the delivery efficiency of the drug using three commercially available respiratory drug delivery platforms: a disposable DPI (TwinCaps®), a vibrating mesh nebulizer (Aerogen Solo®) and a nasal spray (VP7 Aptar®). The composition of spray dried NIC-hLYS particles was optimized using a constrained-mixtures design of experiments (DoE) to achieve respirable dry particles (geometric median diameter ≤5 μm) that could be easily reconstituted as suspension suitable for nasal spray or nebulizer-based administration. The DoE generated several powder formulations (Table 1), of which formulation 8 (F8) was selected for further characterization

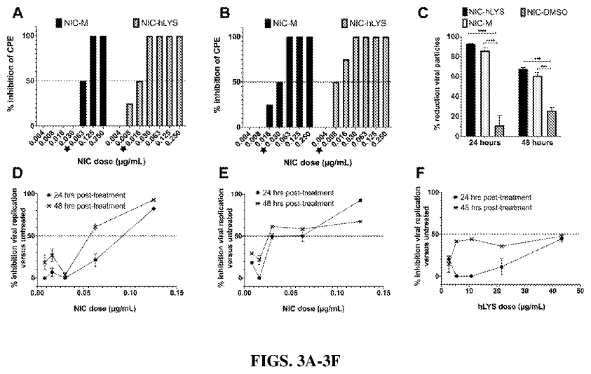

For ambulatory patients, DPIs provide a convenient treatment option for lung-targeted delivery. The rapid administration time for the device as well as the compact size improves patient acceptability and compliance. A disposable DPI is likely preferred in the treatment of COVID-19, given the currently unknown risks regarding re-infectability. A commercially available disposable DPI, the TwinCaps® (Hovione) was therefore selected a model delivery platform. NIC-hLYS powder inhalation was successfully delivered using the TwinCaps DPI, with a 136.0±7.4 μg NIC fine particle dose (i.e., recovered drug mass with less than 5 μm aerodynamic diameter) achieved per 60 mg powder actuation (0.7% NIC content) when using flow rate conditions reflective of a healthy patient (FIG. 3H). Given the potential for shortness of breath and reduced inspiratory abilities that may occur in COVID-19, the delivery efficiency was also examined using flow-rate conditions reflective of a patient with reduced lung capacity due to illness or age. Similar fine particle doses (121.1±7.7 μg) were achieved in these reduced inhalation flow rate conditions, which indicates a minimal dependence on inspiratory flow rate to achieve successful delivery of the drug to the peripheral lung regions.

COVID-19 may result in the need for mechanical ventilation for continued patient survival. The delivery efficiency of reconstituted NIC-hLYS particles was assessed using an Aerogen Solo vibrating mesh nebulizer, which can be utilized aerosol drug delivery in-line with a ventilator circuit. NIC-hLYS powder reconstituted in 0.45% sodium chloride to a 25 mg/mL concentration (equivalent to 175 μg/mL NIC) resulted in the delivery of a fine particle dose of 62.3±6.4 μg NIC after a 2-minute run time (FIG. 3H). Furthermore, a range of concentrations (10 to 100 mg/mL) could be successfully emitted using the Aerogen Solo device (FIG. 3G). By altering the reconstitution concentration, the dose of NIC-hLYS could therefore be adjusted if required for pediatric patients, or those with hepatic or renal insufficiencies. The zeta potential of the reconstituted NIC-hLYS powder was determined to be +1.8, in contrast to the poorly performing NIC-BSA particles, which exhibited a zeta potential of −10.9 when reconstituted in water.

Epithelial cells of the upper respiratory tract (i.e., nasal passages) exhibit significantly higher expression of ACE2 receptors than those of the lower respiratory tract, which indicates these cells may be more prone to infection with SARS-CoV-2 (Hou et al., 2020). As such, the feasibility of administration of the NIC-hLYS formulation using a nasal spray was assessed using plume geometry analysis. NIC-hLYS powders were reconstituted in 0.45% sodium chloride at concentrations ranging from 10 to 50 mg/mL and actuated using a VP7 Aptar® nasal spray device. Suitable plume angles and uniform spray patterns for nasal administration were achieved for all tested concentrations (FIG. 3I). An increase in plume angle was noted at increasing powder concentrations which was inversely related to changes in suspension viscosity (Table 4) and may be reflective of the decreased surface tension resulting from the presence of hLYS and other surface-active stabilizers. The utilization of a slightly hypotonic reconstitution medium (Table 2) was selected to improve absorption and nose-to-brain penetration of NIC-hLYS. The decreases in brain and kidney viral titers noted in vivo when NIC-hLYS was administered intranasally may reflect an achievement of therapeutic drug concentrations outside the respiratory passageways.

TABLE 4
Spray pattern analysis of varying concentrations of NIC-hLYS emitted
from the VP7 Aptar ® nasal spray device.

| | | Spray Pattern 2 cm | | | Spray Pattern 5 cm | | |
|---|---|---|---|---|---|---|---|
| | Plume | Spray | Max | Min | Spray | Max | Min |
| Concentration | Angle | Area | Diameter | Diameter | Area | Diameter | Diameter |
| (mg/mL) | (°) | (mm²) | (mm) | (mm) | (mm²) | (mm) | (mm) |
| 10 | 42.3 ± | 432 ± | 24.4 ± | 23.4 ± | 1398 ± | 49.6 ± | 38.0 ± |
| | 1.4 | 8.7 | 0.3 | 0.2 | 123.7 | 1.9 | 2.4 |
| 25 | 45.5 ± | 434 ± | 26.7 ± | 22.9 ± | 1799 ± | 58.8 ± | 43.6 ± |
| | 4.7 | 9.9 | 1.9 | 0.2 | 151.5 | 1.5 | 1.8 |
| 50 | 58.6 ± | 470 ± | 28.5 ± | 23.4 ± | 2668 ± | 64.0 ± | 55.1 ± |
| | 2.8 | 3.8 | 0.5 | 0.25 | 1025.1 | 14.0 | 9.7 |

NIC is a poorly water-soluble drug, which renders the commercially available oral formulation ineffective against respiratory diseases due to the limited absorption of the drug from the gastrointestinal tract. Direct delivery to the airways represents a promising alternative to oral delivery, as it would enable achievement of high levels of drug at the site of disease. However, limited solubility and delayed dissolution of niclosamide particles in the upper respiratory tract could result in rapid clearance of the particles by the mucocilliary escalator or through alveolar macrophage uptake. hLYS exhibits surface active properties which could enhance the dissolution rate of poorly water soluble NIC particles. To test this hypothesis, the dissolution rate of NIC-hLYS particles exhibiting an aerodynamic diameter of ˜2 μm was compared against hLYS-free NIC particles in simulated lung fluid medium. Though the deposited particles exhibited equivalent sizes and surface areas, the inclusion of hLYS as a formulation component resulted in a slightly faster rate of dissolution, with 82.1% the deposited dose of NIC dissolved by 6 hours (FIG. 3F).

### Example 3—Therapeutic Applications of Compositions

### A. Coronaviruses

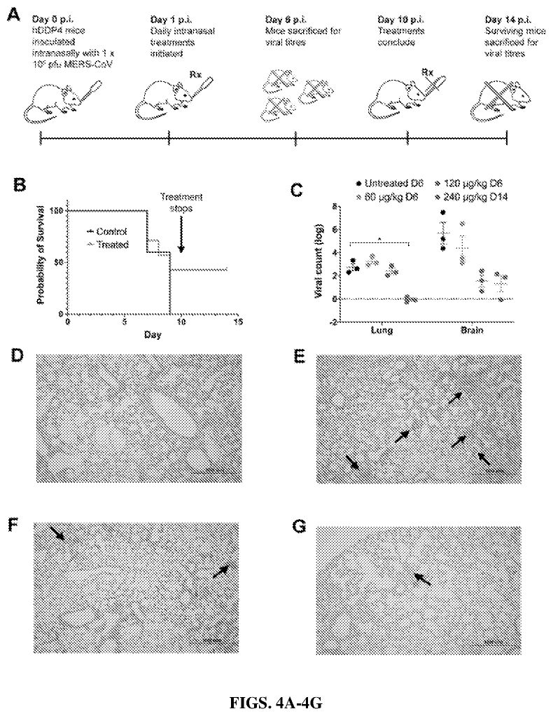

To determine the utility of endogenous hLYS as a therapeutically active carrier molecule for the nasal and pulmonary delivery of NIC, the in vitro antiviral activity of the pharmaceutical composition was assessed against a lysozyme-free NIC suspension. NIC-hLYS particles (0.7% w/w NIC) were administered at varying doses (based upon NIC content) to Vero E6 cells infected with MERS-CoV or SARS-CoV-2, and the EC₅₀ was calculated based upon observed CPE. The addition of hLYS to the NIC formulation resulted in improved anti-viral activity based upon reductions in the EC₅₀ dose for MERS-CoV (0.016 μg/mL NIC to 0.0625 μg/mL NIC) and SARS-CoV-2 (0.030 μg/mL to 0.008 μg/mL) (FIGS. 4A & 5A). A separate assay utilizing qPCR quantification of viral RNA collected from infected cells, the administration of NIC-hLYS particles to Vero cells with established CoV infections resulted in 82.2% viral inhibition relative to untreated controls at 24 hours and 92.7% inhibition by 48 hours for MERS-CoV, and 92.7% inhibition at 24 hours and 67.5% inhibition at 48 hours in SARS-CoV-2 (FIGS. 4D & 5E). Interestingly, hLYS alone also appeared to exhibit activity against SARS-CoV-2 (FIG. 4F), which has not been previously reported. Though the inhibitory activity was not as potent as that of NIC, it may contribute to observed increase in potency of NIC-hLYS in the CPE-based EC₅₀ assay. NIC-hLYS, micronized NIC alone (NIC-M), and NIC dissolved in DMSO were compared for their inhibitory activity against SARS-CoV-2 at a NIC dose of 0.125 μg/mL. No significant differences were noted between quantified viral particles recovered from cells treated with NIC-hLYS or NIC-M, however, both formulations exhibited significantly improved activity compared to solubilized NIC (FIG. 4C). Thus, an improvement in solubility does not appear to be the mechanism for the increased activity of NIC-hLYS.

A separately conducted viability assay in uninfected Vero E6 cells determined that the highest dose of NIC-hLYS utilized (0.125 μg/mL) had no effect on cell viability versus untreated controls (98.3% viability in treated cells).

i. Intranasal Administration of NIC-hLYS Particles to CoV-Infected Mice Improves Survivability and Reduces Viral Loads in Lungs, Brain and Kidneys

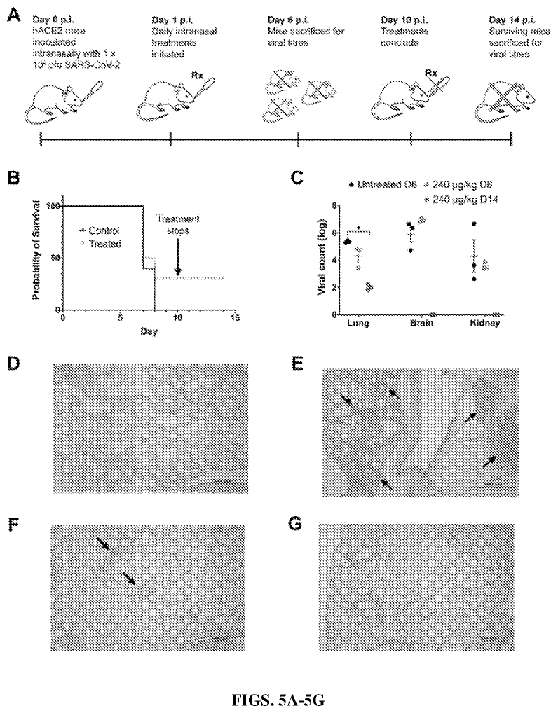

The in vivo efficacy of NIC-hLYS particles was assessed in lethal infection models for both MERS-CoV and SARS-CoV-2. HDDP4 transgenic mice were inoculated intranasally with MERS-CoV (1×10⁵ pfu) and rested for 24 hours, after which once daily treatment with varying doses of intranasal NIC-hLYS (dosed based NIC component) was initiated. In this initial efficacy, animals were sacrificed at Day 6 to determine viral titres in brain and lung tissue compared to untreated controls. Notable decreases in brain viral titres was observed at the 120 μg/kg dose although these differences were not statistically significant (Two-way ANOVA with Dunnet's multiple comparisons test, p=0.1724) (FIG. 5B). In a follow-up survival study utilizing lethal inoculum (1×10⁵ pfu), MERS-CoV-infected hDDP4 mice were dosed at 240 μg/kg NIC-hLYS daily by the intranasal route. By Day 10 (study endpoint), 43% of treated mice had survived compared to 0% of untreated controls (FIG. 5A). Surviving mice were left untreated for an additional 3 days, at which point they were sacrificed. During this period of no treatment, the survival percentage remained at 43%. A statistically significant decrease in lung viral titres was noted in these surviving mice compared to the Day 6 untreated controls from the earlier efficacy study (Two-way ANOVA with Dunnett's multiple comparisons test, p=0.011). While brain viral titres did not exhibit further reduction from levels noted in the preliminary efficacy study, the inoculation of Vero E6 cells with viral particles obtained from lung and brain homogenates of surviving animals resulted in no observation of CPE at any of the inoculum concentrations tested, which indicates that remaining viral particles were not active. Thus, in the 43% of surviving animals it appears that the lethal MERS-CoV infection was essentially cured. Serological assays revealed that surviving animals did express anti-MERS-CoV IgG antibodies.

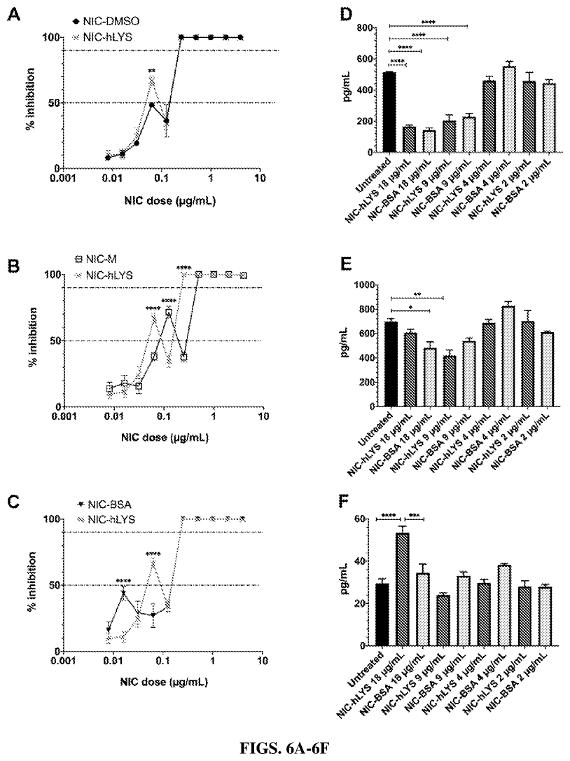

In a similar study, the efficacy of NIC-hLYS particles was assessed in hACE2 transgenic mice infected intranasally with a lethal dose of SARS-CoV-2 (1×10⁴ pfu). After a 24-hour rest period, daily intranasal treatment was 0.9% sodium chloride (untreated control) or NIC-hLYS (240 μg/kg NIC) was initiated. A portion of mice from each group (n=3) were sacrificed at day 6 post-infection to determine viral loads in brain, kidney and lung tissues, while remaining animals were utilized in a 10-day survival study. Non-significant changes in day 6 viral titers were observed (Two-way ANOVA with Dunnet's multiple comparisons test) (FIG. 6B). By the day 10 study endpoint, 30% of treated mice had survived, compared to 0% in the untreated arm (FIG. 6A). Similar to the MERS-CoV study, these surviving mice were left untreated for an additional 3 days, at which point they were sacrificed (Day 14 p.i.). The surviving mice exhibited a statistically significant decrease in viral loads in lung tissue compared to Day 6 p.i. untreated controls (P=0.023, ANOVA with Dunnett's post-hoc analysis), and no virus particles were detected in brain and kidney tissue. Inoculation of Vero E6 cells with 10-fold dilutions of tissue homogenates resulted in no observed CPE, and surviving animals were sera positive for anti-SARS-CoV-2 antibodies.

In both MERS-CoV and SARS-CoV-2 infection models, the lung tissue of infected and NIC-hLYS-treated mice (FIGS. 5F & 6F) showed lower levels of interstitial pneumonia than that of infected and non-treated mice (FIGS. 5E & 6E) on day 6 p.i. Inflammation was further reduced in the treated/infected groups on Day 14 p.i. (FIGS. 5F & 6F) and was more comparable to the mock-infected mice (FIGS. 5D & 6D), which showed no signs of interstitial pneumonia.

ii. Niclosamide-Lysozyme Particles Protect Against Two COVID-19 Sequalae: Secondary Bacterial Infection and Inflammatory Response

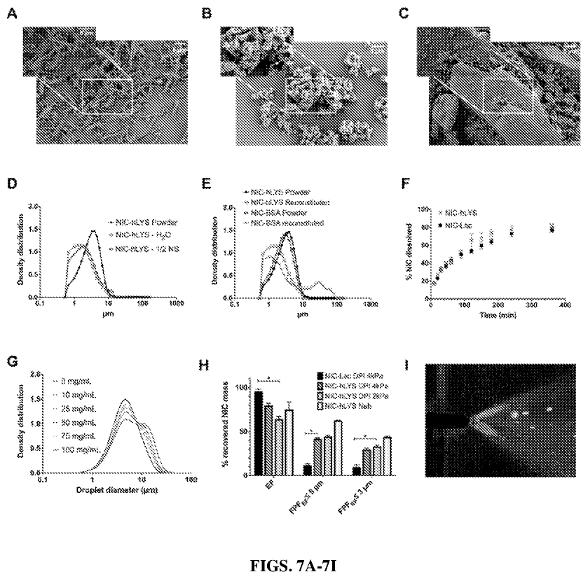

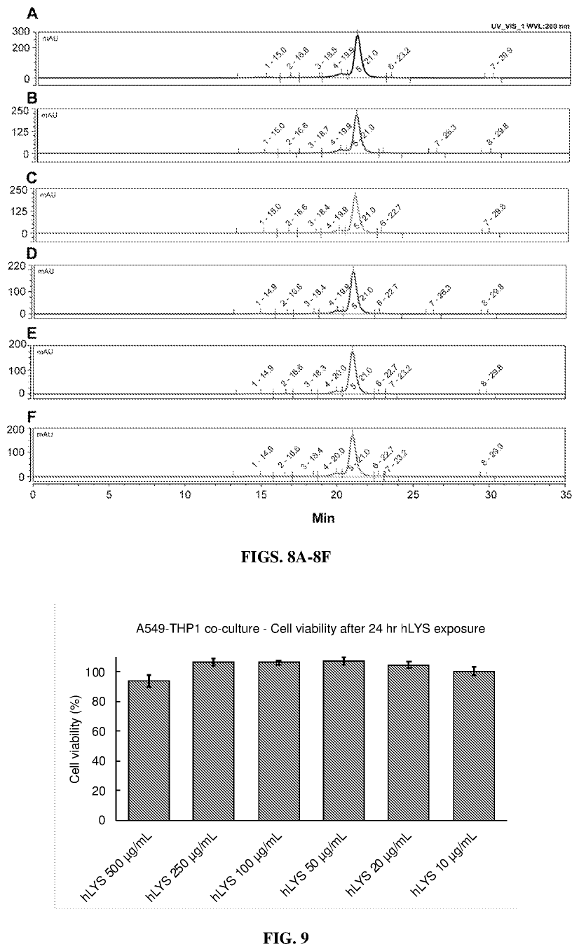

COVID-19 patients may be at-risk for secondary bacterial pneumonias and severe inflammatory lung damage. The efficacy of NIC-hLYS in the treatment of these important COVID-19 sequalae was therefore assessed. A resazurin-based microbroth dilution assay was performed to determine the inhibitory activity of several NIC formulations (NIC-hLYS, NIC-BSA, NIC-M, and NIC-DMSO). Compared to the other NIC formulations, NIC-hLYS reached an MIC50 at lower levels of NIC (0.0625 μg/mL), and 100% inhibition was noted at a concentration of 0.125 μg/mL (FIGS. 7A-7C). No inhibitory activity was observed for hLYS alone. Interestingly, all NIC formulations exhibited a similar dose-response profile where a sharp dip in activity preceded the concentrations at which 100% inhibition was achieved. This same pattern was also noted in the dose-response profiles for anti-MERS-CoV and anti-SARS-CoV-2 activity (FIGS. 4D & 4E). Plating of the wells with 100% inhibition noted resulted in the growth of colonies, which indicates that the antimicrobial activity of NIC may be bacteriostatic rather than bactericidal.

A feared consequence of SARS-CoV-2 infection is the occurrence of ARDS, which is a major contributor to morbidity and mortality and dramatically increases the burden on healthcare systems. ARDS is caused by the massive release of inflammatory cytokines in the lungs, which occurs in some patients in response to pathogenic infiltration. Both NIC and hLYS are known to exhibit anti-inflammatory activity. The anti-inflammatory activity of two NIC formulations, NIC-BSA and NIC-hLYS, was assessed using an acute macrophage inflammation model. NIC-hLYS and NIC-BSA exhibited similar suppression of the inflammatory cytokines IL-6 and TNF-a (FIGS. 7D & 7E). The similarities in the levels of these two cytokines across the two formulations tested indicates that the suppression may be related to the activity of NIC rather than hLYS. Suppression of IL-1b was not observed for either formulation, and the highest concentration of NIC-hLYS tested (18 μg/mL total powder) resulted in statistically significant increase in compared to both the untreated, LPS-stimulated control and an equivalent dose of the NIC-BSA formulation (FIG. 7F).

### B. Cancer

The efficacy of a niclosamide-lysozyme combination was tested against an adenocarcinomic human alveolar epithelial cell line (A549). To better simulate the lung environment, a mixed culture model of A549 cells and macrophage cells (differentiated THP-1 cells) was utilized. A549 and THP-1 cells (previously differentiated to macrophages using 48 hours incubation in the presence of 10 ng/mL PMA) were co-cultured in liquid in a 10:1 ratio (initially plated at 5:1 ratio, as A549 cells continue to divide while differentiated THP-1 do not). The cells were rested for 24 hours, then exposed to varying doses of micronized niclosamide, human lysozyme or a combination of the two for 24 hours, after which an MTT assay was performed. The absorbance of the cells at 570 nm was normalized to the average of the absorbance of the untreated cells to determine the effect of the treatments on cell viability. Treatments were administered to the cells.

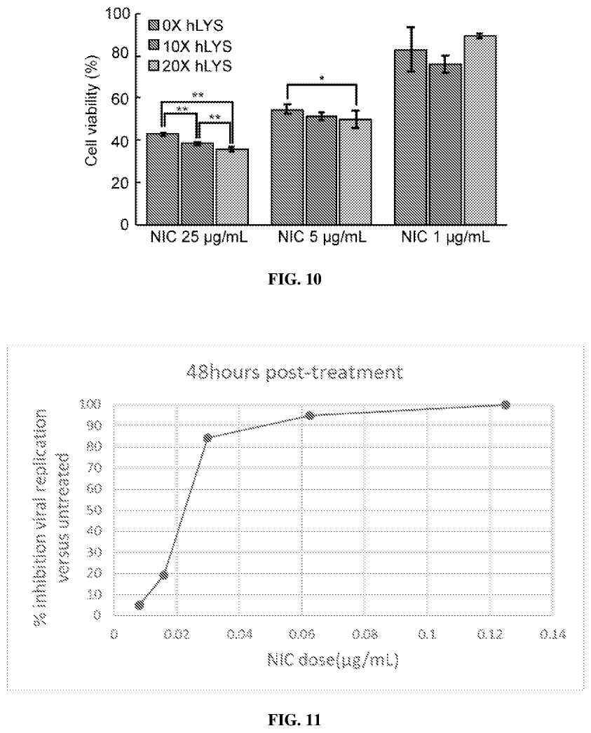

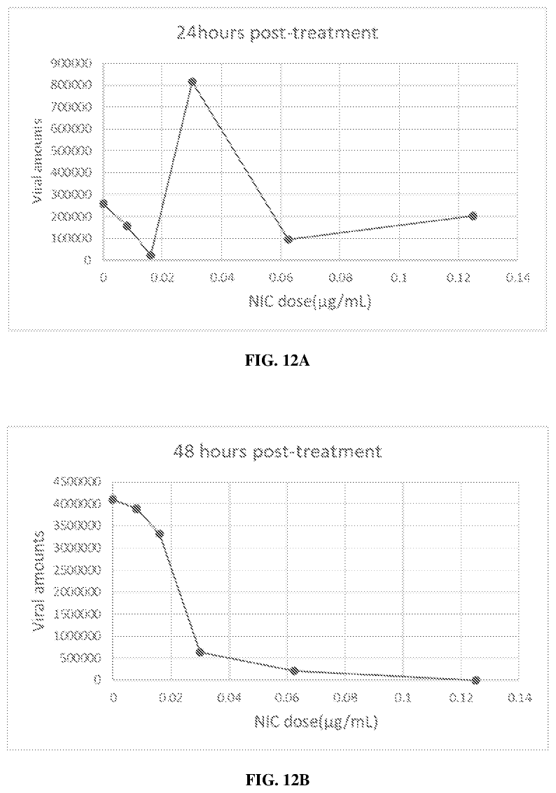

Human lysozyme did not have an effect on the viability co-cultured cells until a concentration of 500 μg/mL. The effect at this concentration was minimal (93.8% viability, normalized to untreated control) (FIG. 9). Co-administration of human lysozyme did not appear to have an inhibitory effect on the toxicity of NIC micronized particles, which may indicate that this pharmacological effect is not dependent upon protein binding. Lysozyme appeared to enhance the toxicity at higher NIC doses, which may be related to an enhanced dissolution rate or solubility of NIC in the presence of the lysozyme (FIG. 10).

### C. Respiratory Syncytial Virus (RSV) Infection

NIC-hLYS particles were treated to RSV infected Hep-2 cells in 5 different concentrations (0.008, 0.016, 0.03, 0.0625, 0.125 μg/mL). No antiviral effects were shown on 24-hours of exposure to NIC-hLYS particles, but significant RSV inhibition effects were shown on 48-hours of exposure. We assessed the antiviral dose-response of NIC-hLYS particles in a separate assay utilizing qPCR quantification of viral RNA collected from infected cells. A dose 0.125 μg/mL NIC resulted in a 48-hour inhibition of 99.9% relative to untreated controls. But further research is needed since high dose of NIC could lead to cell death. A dose of 0.0625 μg/mL NIC resulted in a 48-hour inhibition of 94.7% relative to untreated controls (FIGS. 11 & 12).

TABLE 5
Change in Inhibition Percentage as a Function of Concentration

| | % inhibition viral replication |
|---|---|
| Concentration (μg/mL) | versus untreated: 48 hours |
| 0.008 | 5.126265 |
| 0.016 | 19.26527 |
| 0.03 | 84.36796 |
| 0.0625 | 94.6989 |
| 0.125 | 99.85859 |

### Example 4—Methods

### A. Engineering and Optimization of Inhalable Composite Particles of Niclosamide and Human Lysozyme

Niclosamide (NIC) was obtained from Shenzhen Neconn Pharmtechs Ltd. (Shenzhen, China) and micronized in-house using an Model 00 Jet-O-Mizer air jet mill (Fluid Energy Processing and Equipment Co, Telford, PA, USA) using a grind pressure of 75 PSI and a feed pressure of 65 PSI for a total of three milling cycles to achieve an X50 diameter of 2.2 μm and an X90 diameter of 4.1 μm. To generate a powder formulation of NIC suitable for DPI-based delivery as well as suspension-based nebulizer and nasal spray delivery, micronized NIC particles were embedded in a matrix of recombinant human lysozyme (hLYS) (InVitria, Junction City, KS, USA), sucrose (Sigma-Aldrich, Darmstadt, Germany), polysorbate 80 (Sigma-Aldrich) and histidine (Sigma-Aldrich) using spray drying. Histidine (buffering agent), sucrose (lyoprotectant agent), and polysorbate 80 (surface active agent) can be used to generate stable and dispersible formulations of lysozyme for delivery via DPI (Brunaugh et al., 2019; and Brunaugh et al., 2017).

Preliminary screening experiments indicated that spray drying with a feed solid content greater than 1% w/v resulted in a dry particle size distribution (PSD) that was greater than the respirable size (typically less than 5 μm particle diameter). A constrained mixtures DoE was therefore utilized to determine the optimal ratio of micronized niclosamide, human lysozyme, sucrose, and polysorbate 80 in the 1% w/v feed to generate powder with both a dry and reconstituted PSD suitable for oral or nasal inhalation (Table 6).

TABLE 6
Upper and lower constraints for mixture DoE

| Component | Lower constraint (% w/w) | Upper constraint (% w/w) |
|---|---|---|
| Micronized niclosamide | 0.3 | 1 |
| Human lysozyme | 60 | 80 |
| Sucrose | 20 | 40 |
| Polysorbate 80 | 0.05 | 0.2 |

| | |
|---|---|
| Histidine | Fixed at 0.174 mg/mL in feed solution |

The dry components of the formulations were mixed using a process of geometric dilution and wetted and suspended using polysorbate 80 followed by incremental additions of 0.174 mg/mL histidine buffer. All suspensions were spray dried with a BUCHI B-290 mini-spray dryer (BUCHI Corporation, New Castle, DE, USA) coupled to a syringe pump (KD Scientific Inc, Holliston, MA, USA) set at a feed rate of 1 mL/min A 2-fluid pneumatic atomizer nozzle (0.7 mm with 1.5 mm cap) was used to atomize the suspension, and house air was used as the atomization gas. The cleaning needle of the nozzle was removed to prevent disruptions to the feed flow rate (Brunaugh et al., 2019). The spray dryer was set at an inlet temperature of 130° C., which corresponded to an outlet temperature of ˜70° C. For all runs, no settling of the feed suspensions was noted during processing. Formulations were evaluated on the basis of dry powder PSD and reconstituted suspension PSD, and the composition exhibiting the most promising characteristics was selected for further evaluation. Comparative powders were generated for the purposes of evaluation of physicochemical characteristics, aerosol performance, and efficacy. A NIC-free hLYS spray dried powder was generated using the optimized formulation composition identified in the DoE, minus the addition of micronized NIC. To compare the novel NIC-hLYS powders against a traditional lactose-based carrier system, micronized NIC was blended with crystalline lactose particles (Lactohale 100; DFE Pharma) using geometric dilution followed by mixing in a Turbula powder blender. The concentration NIC in the niclosamide-lactose blend (NIC-Lac) was set to match that in the NIC-hLYS powder (0.7%). Lastly, a powder was generated in which bovine serum albumin (BSA) was substituted for the hLYS in the optimized NIC formulation, in order to assess the effect of the protein on formulation characteristics.

TABLE 7
D-optimal subset utilized for constrained mixtures DoE

| | | | | X4 | |
|---|---|---|---|---|---|
| | | | | (polysorbate | |
| Run | X1 (NIC-M) | X2 (hLYS) | X3 (sucrose) | 80) | Dimension |
| 1 | 0.0030 | 0.6000 | 0.3965 | 0.0005 | 0 |
| 2 | 0.0100 | 0.6000 | 0.3895 | 0.0005 | 0 |
| 3 | 0.0030 | 0.7965 | 0.2000 | 0.0005 | 0 |
| 4 | 0.0100 | 0.7895 | 0.2000 | 0.0005 | 0 |
| 5 | 0.0030 | 0.6000 | 0.3950 | 0.0020 | 0 |
| 6 | 0.0030 | 0.7950 | 0.2000 | 0.0020 | 0 |
| 7 | 0.0100 | 0.6000 | 0.3880 | 0.0020 | 0 |
| 8 | 0.0100 | 0.7880 | 0.2000 | 0.0020 | 0 |
| 14 | 0.0100 | 0.7888 | 0.2000 | 0.0012 | 1 |
| 21 | 0.0030 | 0.6979 | 0.2979 | 0.0012 | 2 |
| 23 | 0.0065 | 0.6000 | 0.3922 | 0.0012 | 2 |
| 25 | 0.0065 | 0.6965 | 0.2965 | 0.0005 | 2 |

### B. Physicochemical Characterization of Niclosamide-Lysozyme Composite Particles

Particle size distribution (PSD) of NIC-hLYS powders was measured using a RODOS disperser coupled to a Sympatec laser diffractor unit (Sympatec GmbH, Clausthal-Zellerfeld, Germany) Dispersion pressure was set at 3.0 bar and feed table rotation was set at 20%. Time slices of the plume exhibiting an optical concentration between 5-25% were averaged to generate the PSD. The PSD of the reconstituted powders was determined in both ½ normal saline (NS) and DI water using the Cuvette attachment for the laser diffraction. A spin bar was set to rotate at 2000 RPM, and the dry powders were added directly to the solvent in the cuvette until an optical concentration exceeding 5% was reached. Three measurements were taken and averaged. Zeta potential of NIC-hLYS suspensions before and after spray drying was determined using a Zetasizer NanoZS (Malvern Panalytical Ltd, Malvern, UK) and compared against a NIC-BSA suspension.

The morphology of NIC-hLYS powders was observed using scanning electron microscopy (SEM). Samples were mounted onto aluminum stubs using double-side carbon tape and sputter coated with 15 nm of platinum/palladium (Pt/Pd) under argon using a Cressington sputter coater 208 HR (Cressington Scientific Instruments Ltd, Watford, UK). Images were obtained using a Zeiss Supra 40VP SEM (Carl Zeiss Microscopy GmbH, Jena, Germany)

Glass transition temperature (Tg) and crystallinity of NIC-hLYS powder was determined using modulated DSC. Powder samples were loaded into Tzero pans with hermetically sealed lids, and a hole was pierced to prevent pan deformation. A scan was performed on a Q20 DSC (TA Instruments, New Castle, DE, USA) by ramping 10° C./min to −40° C., followed by a 2° C./min ramp from −40° C. to 280° C. with a modulation cycle of ±0.5° C. every 40 seconds. Data was processed using TA Universal Analysis.

The dissolution profile of the aerosolized NIC-hLYS and NIC-Lac powders was evaluated based upon previously published methods (64, 65) using a composition of simulated lung fluid (SLF) adapted from Hassoun et al (66). Briefly, Whatman GF/C glass microfiber filters (diameter=24 mm) were placed in a Stage 4 of a Next Generation Impactor, which corresponded to an aerodynamic size cut off of 2.01 μm at the 40 L/min flow rate used in the experiment. Powders were dispersed using a disposable TwinCaps (Hovione) DPI. The filters were transferred to a modified Transwell system (membrane removed) to enable contact of the bottom of the filter with a basal compartment containing 1.5 mL SLF. The apical side of the filters were wetted with 0.1 mL of SLF. The dissolution results are presented in Table 3. The Transwell system was placed in a 37° C. isothermal chamber and 0.1 mL samples were removed from the basal compartment at various timepoints and replaced with fresh SLF.

### C. Stability Analysis of Human Lysozyme

Effects of processing and nebulization on the aggregation of hLYS was assessed using size exclusion chromatography (SEC) based upon a previously published method (60). The effect of processing on the secondary structure of hLYS was determined using a Nicloet iS50 Fourier transform infrared spectrophotometer with attenuated total reflectance (FTIR-ATR) (Thermo Scientific). Spectra were acquired using OMNIC software from a wavelength of 700 cm⁻¹ to 4000 cm⁻¹ with 64 acquisitions in total. An atmospheric background scan was collected and subtracted from all powder spectra. Secondary structure analysis was performed in OriginPro (OrginLab Corporation) using the second derivative of the amide I band region (1580-1720 nm) and the peak analyzer function. The region of interest was first baseline-corrected, and the second derivative of the spectra was smoothed using the Savitzky-Golay method with a polynomial order of 2 and 50 points in the smoothing window. Peaks identified from the second derivative minimums were iteratively removed to assess the effect on the model fit.

### D. Aerosol Performance Testing

The aerosol performance of the spray-dried composite NIC-hLYS powder was assessed using a disposable TwinCaps DPI. Performance was assessed at both a 4 kPa and 2 kPa pressure drop through the device to determine the effects of inspiratory flow rate on emitted and fine particle dose. For comparative purposes, the performance of traditional lactose carrier-based dry powder, NIC-Lac, was also assessed. A 4 kPa pressure drop was generated through the TwinCaps DPI using an inspiratory flow rate of 40 L/min. A 6.5 second actuation time was used to pull 4 L of air through the NGI. A 2 kPa pressure drop was generated using an inspiratory flow rate of 28.3 L/min, and an 8 second actuation time was used. For all experiments, 60 mg of powder was loaded into the device. After actuation of NIC-hLYS powders, niclosamide was collected by dissolving the deposited powder using a 50-50 water:acetonitrile mix. An aliquot was taken, and an additional volume of acetonitrile was added to bring the final ratio to 20:80 water:acetonitrile. To induce phase separation, A 2 M solution of ammonium acetate was added to this mixture at a volume that was 20% of the water:acetonitrile mix. Niclosamide was assayed from the upper organic layer by measuring absorbance at 331 nm using a plate reader. For the niclosamide-lactose blend, the deposited powder was collected by dissolving it in 20:80 water:acetonitrile, centrifuging, and then measuring absorbance at 331 nm.

Delivery of the reconstitued NIC-hLYS suspension was assessed using the disposable Aerogen® Solo vibrating mesh nebulizer (Aerogen). Preliminary screening experiments indicated that a reconstituting 25 mg/mL of NIC-hLYS powder in 0.45% w/v sodium chloride (½ NS) reduced the changes in nebulizer concentration during therapy compared to higher concentrations; therefore, this concentration was utilized for further analysis. The inspiratory flow rate was set for 15 L/min and the apparatus was chilled to 4° C. as specified by the United States Pharmacopeia (USP). Nebulization was performed for two minutes to ensure sufficient deposition of drug in the stages for analysis.

After drug collection, aerosol performance was evaluated on the basis of emitted fraction or dose (niclosamide mass emitted from the device as a percentage of the total recovered powder) and fine particle fraction (niclosamide mass with a size cut off of less than 5 μm aerodynamic diameter or 3 μm aerodynamic diameter, as a percentage of the emitted dose). NGI stage cut-offs were determined for the flow rate utilized based upon Eq. 1, while the MOC cut-off diameter was determined using Eq. 2.

$${D}_{50 , Q} = {{D}_{50 , Qn} ( \frac{{Q}_{n}}{Q} )}^{x} \qquad Eq . 1$$

$${D}_{80 , Q} = 0.14 {( \frac{{Q}_{n}}{Q} )}^{1.36} \qquad Eq . 2$$

Where D_{50,Q} is the cut-off diameter at the flow rate Q, D_{50,Qn} is the cut-off diameter at the archival reference values of Qₙ=60 L/min, and the values for the exponent, x, are those obtained from the archival NGI stage cut size-flow rate calculations determined by Marple et al(68). Aerosol performance was evaluated on the basis of emitted fraction (EF), which is defined as the cumulative mass emitted from the device as a fraction of the recovered mass, fine particle fraction less than 5 μm (FPF_{<5 μm}), defined as the mass less than 5 μm aerodynamic diameter as a fraction of the emitted dose, and the fine particle fraction less than 3 μm (FPF_{<3 μm}). The FPF_{<5 μm} and FPF_{<3 μm} values were interpolated from a graph plotting the cumulative percentage of NIC deposited in a stage against the cut-off values of the stage.

### E. Nasal Spray Characterization

To evaluate the utility of the optimized NIC-hLYS powders for nasal administration, suspensions of varying concentrations (10 mg/mL, 25 mg/mL, and 50 mg/mL) were prepared in ½ NS and placed in a VP7 pump Aptar® pump meter spray device. Spray patterns and plume geometries were evaluated using laser-assisted high speed imaging based on methods previously reported by Warnken et al. (Warnken et al., 2018). Briefly, the loaded spray devices were actuated using a MightyRunt automated actuator (InnovaSystems, Inc) set at parameters that mimic those of an average adult user (Doughty et al., 2011). A laser-sheet was oriented either parallel or perpendicular to the actuated spray at distances of 2 and 5 cm from the nozzle tip in order to assess the plume geometry and spray pattern, respectively. The actuation was conducted in a light-free environment in order to isolate the portions of the spray photographed by the high-speed camera (Thorlabs, Inc.) from those illuminated in the plane of the laser. Image analysis of the plume geometry and spray pattern were performed in Fiji (Schindelin et al, 2012). For the plume geometry, the outline of the observed plume was traced and the slope of each side of the plume was determined. This was used to calculate the angle formed at the intersection of the two lines. The spray pattern characteristics including maximum and minimum diameters were determined using the software's measurement function to determine the ferret diameters.

### F. In Vitro Anti-Inflammatory Efficacy

The ability of the optimized NIC-hLYS formulation to dampen inflammatory response was evaluated using lipopolysaccharide (LPS) stimulation in a THP-1 macrophage model. THP-1 monocytes were seeded in 6-well plates at a concentration of 4×10⁵ cells/mL (5 mL total) in RPMI 1640 media supplemented with 10% FBS, 1% penicillin/streptomycin, and 15 ng/mL phorbol 12-myristate (PMA) to induce differentiation into mature macrophages. The cells were incubated in the presence of PMA for 48 hours, after the media was replaced with PMA-free media and cells were rested for 24 hours. NIC-hLYS and NIC-BSA powders were suspended in RMPI 1640 media at varying concentrations (2 to 18 μg/mL, based on total powder content) and added to the cells simultaneously with 10 ng/mL LPS. The cells were then incubated for 6 hours to achieve peak cytokine expression(72). Following incubation, supernatants were collected, and cytokine concentrations were quantified using ELISA (DuoSet, R&D Systems) and compared against untreated controls.

### G. In Vitro Efficacy

All anti-viral efficacy experiments were performed using Vero-E6 cells obtained from American Type Culture Collection (Manassas, Virginia, USA). Vero-E6 cells were maintained in Minimal Essential Medium (MEM) supplemented with 10% fetal bovine serum (FBS) and 1× antibiotic-antimycotic solution (Sigma, St. Louis, USA) (i.e., MEM complete). Cells were infected with either MERS-CoV (EMC2012 strain) or SARS-CoV-2 (SARS-CoV-2/human/Korea/CNUHV03/2020 strain). All experimental procedures involving potential contact with MERS-CoV or SARS-CoV-2 were conducted in a biosafety level 3 laboratory of Chungnam National University, which was certified by the Korean government.

i. Efficacy Determined by Viral Titer

Vero-E6 cells (2×10⁴/ml) were seeded in the wells of 6-well tissue culture plates. After a 3-day incubation period, cells were washed with warm PBS (pH 7.4) twice and were infected with SARS-CoV-2 (1.7×10³ pfu) or MERS-CoV (2×10⁴ pfu) diluted in MEM with 2% FBS, which was followed by a 24-hour rest period. The media was then replaced with MEM complete containing various concentrations of the investigational formulations (prepared as suspensions). For the assessment of solubilized NIC, stock solutions were prepared by dissolving the drug in DMSO, and the diluting in MEM-complete, with the resulting media not containing more than 1% DMSO. Drug concentrations and formulations were assessed in triplicate. For each 6-well plate, 1 well was utilized as untreated control. Cells were incubated for 24 or 48 hours, at which point viral RNA from samples was isolated using RNeasy Mini Kit (QIAGEN, Hilden, Germany) Viral RNA was quantified with TaqMan real time fluorescent PCR (RTqPCR) using a TOPreal™ One-step RT qPCR Kit (Enzynomics, Daejeon, Korea) and SARS-CoV-2 and MERS-CoV primers and probe (Table 8). Real-time amplification was performed using a Rotor-Gene 6000 (QIAGEN, Hilden, Germany) An initial incubation was performed at 50° C. for 30 minutes and at 95° C. for 10 minutes, after which 45 cycles of a 5 second hold at 95° C. and a 30 second hold at 60° C. were performed. Cycle threshold (Ct) values were converted to plaque forming units (pfu) using a standard curve generated from data using stock viruses with known pfu titers by plaque assay.

TABLE 8
Primer sequences used for the quantification
of viral particles

| Primer | | SEQ ID |
|---|---|---|
| name | Sequence | NO: |
| MERS N3 | GGG TGT ACC TCT TAA TGC | 1 |
| Forward | CAA TTC | |
| MERS N3 | TCT GTC CTG TCT CCG CCA AT | 2 |
| Reverse | | |
| MERS N3 | 5′FAM-ACC CCT GCG CAA AAT | 3 |
| probe | CGT-BHQ1 3′ | |
| SARS-COV-2 | CAC ATT GGC ACC CGC AAT C | 4 |
| Forward | | |
| SARS-COV-2 | GAG GAA CGA GAA GAG GCT TG | 5 |
| Reverse | | |
| SARS-COV-2 | 5′FAM-ACT TCC TCA AGG AAC | 6 |
| Probe | AAC ATT GCC A-BHQ1 3′ | |

ii. EC₅₀ Determination

Vero-E6 cells grown in tissue culture flasks were detached by treatment with trypsin-EDTA and were seeded in 96-well tissue culture plates. When confluent, cells were washed with warm PBS (pH 7.4) and infected with MERS-CoV or SARS-CoV-2. The half maximal effective concentration (EC₅₀) of the formulations was assessed by dosing infected Vero E6 cells plated in 96-wells with NIC-hLYS suspensions with NIC content ranging from 0.25 μg/mL to 0.004 μg/mL once daily over the course of 72 hours. Cell viability was determined on day 4 by observing cytopathic effects (CPE) under microscope. The EC₅₀ was calculated as the concentration of NIC resulting in no observable CPE in 50% of the wells. For comparative purposes, the EC₅₀ of micronized NIC without the inclusion of hLYS was also evaluated.

iii. Inhibitory Activity Against S. Aureus

The inhibitory activity of various NIC formulations (NIC-hLYS, NIC-M, NIC-BSA, and NIC-DMSO) against methicillin-resistant S. aureus strain Mu50 was assessed using a resazurin-based 96-well plate microdilution assay (Elshikh et al., 2016). Varying concentrations of the NIC formulations (: 4, 2, 1, 0.5, 0.25, 0.125, 0.0625, 0.0313, 0.0156, 0.008 μg/mL, dosed based on NIC content) were plated with a 5×10⁵ cfu/mL inoculum of S. aureus (n=6 per dose). One column of the plate was used as growth control, i.e., no antibiotics were added, while another column was used as a sterile control, i.e., no bacteria added. The plates were incubated for 24 hours at 37° C. with 150 RPM shaking, after which point 30 μL a 0.015% resazurin sodium solution was added. The plates were incubated for an additional 2 hours to allow color change to occur, and the fluorescence of the wells was read at 530 nm excitation/590 nm emission. The fluorescence of the sterile wells was subtracted from the fluorescence of all treated wells, and a decrease in fluorescence of the treated wells versus the growth control was noted as inhibitory activity. The content of wells exhibiting 100% inhibition were plated on tryptic soy agar plates and incubated overnight to determine the mean bactericidal concentration (MBC).

### H. In Vivo Efficacy Assessment

i. MERS-CoV

hDPP-4 transgenic mice were kindly provided by Dr. Paul B. McCray Jr (University of Iowa). MERS-CoV infection was initiated in anaesthetized mice by intranasal (i.n.) administration of 50 μL (1×10⁵ pfu) of MERS-CoV (EMC2012 strain), which was kindly

Drs Bart Haagmans and Ron Fouchier (Erasmus Medical Center). Efficacy was initially established using a dose- finding study, in which treatments were initiated 1-day post-infection (p.i.) and daily for 6 days, at which point animals (n=3 from each group) were sacrificed. NIC-hLYS powder was reconstituted in 0.45% sodium chloride to achieve a dose of 60 or 120 μg/kg NIC (n=7 per group). The suspensions were administered i.n. in a volume of 50 μL, and 50 μL of 0.9% sodium chloride was administered as a control (n=6). Though a second timepoint was intended for day 9, death due to illness or as a result of treatment administration prevented obtainment of these data. A separate study was conducted to compare the survival of MERS-CoV-infected mice treated with 240 μg/kg NIC-hLYS (n=7) and placebo (n=6). In this study, mice were dosed intranasally for 10 days, at which point treatment was terminated. Surviving mice were rested without treatment for an additional 3 days, and sacrifice was performed Day 14 p.i. to obtain tissues (lung and brain) for viral titres and tissue pathology. The weight of mice was recorded daily. Tissues (0.1 g per sample) were homogenised using a BeadBlaster homogeniser (Benchmark Scientific, Edison, New Jersey, USA) in 1 mL of PBS (pH 7.4) to measure virus titres by RT-qPCR. The remaining portions of tissues were used for histopathology. Mice were lightly anaesthetized with isoflurane USP (Gujarat, India) prior to all viral inoculation and dosing procedures.

ii. SARS-CoV-2 Infection

hACE-2 transgenic mice (K18-hACE2 mice) (The Jackson Laboratory, USA) were lightly anaesthetized with isoflurane USP (Gujarat, India) and inoculated intranasally (i.n.) with 50 μL (1×10⁴ pfu) of SARS-CoV-2/human/Korea/CNUHV03/2020. Animals were rested for 24-hours, after which daily treatment was initiated with i.n. NIC-hLYS reconstituted in 0.45% sodium chloride (240 μg/kg NIC) (n=13) or 0.9% sodium chloride as a placebo (n=8). All treatments were performed on anaesthetized mice. On day 6 post-infection, 3 mice per group were euthanized, and lung, brain and kidney tissues were collected for viral titres and tissue pathology. Treatment was performed until 10 days p.i., at which point surviving animals were left untreated for 3 days, and then sacrificed on day 14 p.i. to obtain tissues for viral titres and pathology. Tissues (0.1 g per sample) were homogenised using a BeadBlaster homogeniser (Benchmark Scientific, Edison, New Jersey, USA) in 1 mL of PBS (pH 7.4) to measure virus titres by RT-qPCR. The remaining portions of tissues were used for histopathology.

iii. Preparation of Tissues for Histopathology

Mouse tissues were fixed in 10% neutral buffered formalin (10%) and then embedded in paraffin. The lung tissue was cut into 5 μm sections, which were stained with haematoxylin (H) solution for 4 min. The stained tissue sections were washed with tap water for 10 min and then stained with eosin (E) solution. The stained sections were visualised under an Olympus DP70 microscope and photographed (Olympus Corporation, Tokyo, Japan).

iv. TCID₅₀ Assay

To determine whether measured viral particles in lung and brain tissue were dead or alive, the log₁₀TCID₅₀/mL was determined. Vero-E6 cells grown in tissue culture flasks were detached by treatment with trypsin-EDTA and were seeded in 96-well tissue culture plates with MEM containing 10% FBS and 1× antibiotic-antimycotic solution. When confluent, the cells were washed with warm PBS (pH 7.4) and infected with virus samples, which were 10-fold diluted in MEM with 2% FBS. The cells in four wells were infected with the diluted virus samples for 4 days in a humidified incubator at 37° C. The cells were observed for CPE under microscope.

v. Antibody Detection

The presence of IgG antibody specific for MERS-CoV or SARS-CoV-2 in the sera of infected and treated animals was determined using enzyme-linked immunosorbent assays (ELISA). The purified and inactivated MERS-CoV or SARS-CoV-2 antigen was diluted to final concentration of 100 μg/ml in coating buffer (carbonate-bicarbonate buffer, pH 9.6). The diluted antigen (100 μl) was coated to the wells of a Nunc-Immuno™ MicroWell™ 96 well solid plates (Sigma-Aldrich, MO, USA) and was incubated overnight at 4° C. After removing the coating buffer, the plate was washed twice by filling the wells with 400 μl of washing buffer (0.05% tween 20 PBS (pH 7.4) containing 4% horse serum). To block the remaining protein-binding sites, 400 μl of blocking buffer (PBS containing 4% skim milk) was added to the plate and incubated overnight at 4° C. The buffer was removed, and sera (100 μL diluted in 1:64 in PBS) collected from treated mice on 14 days post treatment were added to the plate and incubated for 1 hr at room temperature. The plate was washed 4 times with washing buffer. Goat anti-Mouse IgG Cross-Adsorbed Secondary Antibody HRP (Invitrogen, MA, USA) was diluted (1:5000) in blocking buffer, and 100 μL was added to each well and incubated for 1 hr at room temperature. After washing the plate 4 times with the washing buffer, 100 μL of the TMB ELISA substrate (Mabtech, Nacka Strand, Sweden) was dispensed into the wells and incubated for 30min at 4° C. ABTS® Peroxidase Stop Solution (KPL, MD, USA) (100 μL) was then added to the plate. The absorbance of each well was measured at 450 nm using iMARK™ Microplate Absorbance Reader (Bio-Rad, CA, USA).

vi. Respiratory Syncytial Virus (RSV) Infection

Hep-2 cells were maintained in Dulbecco's Minimal Essential Medium (DMEM) supplemented with 10% fetal bovine serum (FBS) and 1× antibiotic-antimycotic solution (Sigma, St. Louis, USA) (i.e., MEM complete). Cells were infected with RSV (ATCC VR-1580) using DMEM with 2% FBS. The experiment was conducted in a BSL-2 facility at the University of Texas at Austin.

Hep-2 cells (1×10⁶) were seeded in the wells of 6-well tissue culture plates. After 24 h of incubation, cells were washed with warm DPBS and infected with RSV (2×10⁴ pfu) for 2 hours, followed by a 24-hour rest period. After the rest period, the cell media was replaced with 5 different concentrations of NIC-hLYS solution which is prepared by dissolving different weights of NIC-hLYS in DMEM medium. For each plate, 1 well was replaced with fresh cell medium without drug as untreated control. Cells were incubated with 24 or 48 hours time points, and at each point viral RNA from each well was isolated with RNeasy Mini Kit (QIAGEN, Hilden, Germany) Quantification of viral RNA was performed using One-Step SYBR Green Kit (Invitrogen, Waltham, USA) with RSV primers (F-AGATCAACTTCTGTCATCCAGCAA (SEQ ID NO: 7), R-TTCTGCACATCATAATTAGGAGTATCAAT (SEQ ID NO: 8)). Real-time amplification was performed using aViiA7 (Applied Biosystems, Waltham, USA). An initial incubation was performed at 50° C. for 3minutes and at 95° C. for 5 minutes, after which 40 cycles of a 15 second hold at 95° C. and a 30 second hold at 60° C. were performed. Cycle threshold (Ct) values were converted to plaque forming units (pfu) using a standard curve generated from data using stock viruses with known pfu titers by plaque assay.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Arora et al., Pharm Res, 27(5):786-95, 2010.
Ayerbe-Algaba et al., Frontiers in Cellular and Infection Microbiology, 8(348), 2018.
Barbosa et al., Eur J Pharm Biopharm, 141:58-69, 2019.
Brunaugh et al., Mol Pharm, 14(11):4019-31, 2017.
Brunaugh et al., Molecular Pharmaceutics, 2019.
Cabrita et al., JCI Insight, 4(15), 2019.
Chanput et al., Food Funct, 1(3):254-61, 2010.
Chen et al., Cell Signal, 41:89-96, 2018.
Circu et al., PLoS One, 11(1):e0146931, 2016.
Costabile et al., Mol Pharm, 12(8):2604-17, 2015.
Costabile et al., Molecular pharmaceutics, 12(8):2604-17, 2015.
Domalaon et al., Antimicrobial Agents and Chemotherapy, 63(4):e02574-18, 2019.
Doughty et al., Drug Development and Industrial Pharmacy, 37(3):359-66, 2011.
Elkordy et al., International journal of pharmaceutics, 247(1-2):79-90, 2002.
Elshikh et al., Biotechnol Lett, 38(6):1015-9, 2016.
Ferrari et al., Nature, 183(4660):548, 1959.
Ferrati et al., AAPS PharmSciTech, 19(7):2755-66, 2018.
Fonseca et al., J Biol Chem., 287(21):17530-45, 2012.
Gassen et al., Nat Commun, 10(1):5770, 2019.
Gurwitz, Drug Development Research, 2020.
Gwisai et al., Biomedical Materials, 12(4):045010, 2017.
Hassoun et al., J Drug Deliv Sci Technol, 47:485-91, 2018.
Hinds, Aerosol technology: properties, behavior, and measurement of airborne particles: John Wiley & Sons, 1999.
Hou et al., Cell, 182(2):429-46, 2020.
Ibrahim et al., Journal of Agricultural and Food Chemistry, 59(18):10336-45, 2011.
Imperi et al., Antimicrobial agents and chemotherapy, 57(2):996-1005, 2013.
Inactive Ingredients Database, available from: https://www.accessdata.fda.gov/scripts/cder/iig/index.cfm, 2019.
Inhalation and Nasal Drug Products: Aerosols, Sprays, and Powders—Performance Quality Tests. In: United States Pharmacopeia [Internet].
Ippolito et al., PLoS One, 11(7):e0159675, 2016.
Jeon et al., bioRxiv, 2020
Jurgen et al., PLoS Pathog, 8(10):e1002976, 2012.
Kanth et al., Respir Care, 63(11):1407-12, 2018.
Kissler et al., Science, 368(6493):860-8, 2020.
Koskela et al., Respirology, 10(4):442-8, 2005.
Lai et al., J Microbiol Immunol Infect, 53(3):404-12, 2020.
Lee et al., Inflammation, 38(5):1911-24, 2015.
Lee-Huang et al., Biochemistry, 44(12):4648-55, 2005.
Lenth and Lenth, Vdgraph S. Package ‘rsm’, 2018.
Li et al., Cancer Lett., 349(1):8-14, 2014.
Li et al., JAMA Netw Open, 3(5):e208297, 2020.
Malaczewska et al., BMC Vet Res, 15(1):318, 2019.
Marple et al., Journal of Aerosol medicine, 16(3):301-24, 2003.
Miller et al., Nat Med., 26(8):1212-7, 2020.
Mook et al., Bioorg Med Chem, 23(17):5829-38, 2015.
Moussa et al., J Pharm Sci, 105(2):417-30, 2016.
Peeri et al., International journal of epidemiology, 2020.
Piccaro et al., Antimicrobial agents and chemotherapy, 57(3):1428-33, 2013.
PubChem Database. Niclosamide, CID=4477, Available from: https://pubchem.ncbi.nlm.nih.gov/compound/Niclosamide, 2020.
Qin et al., Clin Infect Dis, 2020.
R Development Core Team. R: A language and environment for statistical computing. Vienna, Austria: R Foundation for Statistical Computing; 2019.
Ragland et al., PLoS Pathog, 13(9):e1006512, 2017.
Rajamuthiah et al., PloS one, 10(4), 2015.
Ratanji et al., J Immunotoxicol, 11(2):99-109, 2014.
Rohrschneider et al., Mol Pharm, 12(8):2618-24, 2015.
Schindelin et al., Nature methods, 9(7):676-82, 2012.
Schweizer et al., PloS one, 13(6):e0198389, 2018.
Sun and Zhang, Tubercle and Lung disease, 79(5):319-20, 1999.
Tam et al., Nat Commun, 9(1):5233, 2018.
Tharmalingam et al., Sci Rep., 8(1):3701, 2018.
Torres et al., 60(10):5663-72, 2016.
Tortorici et al., Nat Struct Mol Biol., 26(6):481-9, 2019.
Travis et al., American journal of respiratory cell and molecular biology, 20(5):872-9, 1999.
Walls et al., Cell, 181(2):281-92, 2020.
Wang et al., Cell research, 30(3):269-71, 2020.
Wang et al., Int J Pharm, 431(1-2):1-11, 2012.
Warnken et al., Molecular Pharmaceutics, 2018.
Weers and Clark, Pharm Res, 34(3):507-28, 2017.
Wen et al., Journal of medicinal chemistry, 50(17):4087-95, 2007.
World Health Organization. WHO Director-General's opening remarks at the Mission briefing on COVID-19, 12 March 2020.
Wu et al., Antimicrobial agents and chemotherapy, 48(7):2693-6, 2004.
Xu et al., ACS Infect Dis., 2020.
Zhang et al., Agricultural Sciences in China, 7(1):112-6, 2008.
Zhurina et al., Microbiology, 86(4):455-62, 2017.

1. A pharmaceutical composition comprising:
(A) niclosamide; and
(B) a protein.

2. The pharmaceutical composition of claim 1, wherein the protein is a protein that is positively charged at physiological pH.

3. The pharmaceutical composition of claim 1, wherein the protein is an immunomodulating protein.

4. (canceled)

5. (canceled)

6. The pharmaceutical composition of claim 1, wherein the protein is lysozyme.

7.-9. (canceled)

10. The pharmaceutical composition of claim 1, wherein the pharmaceutical composition further comprises an excipient.

11. The pharmaceutical composition of claim 10, wherein the excipient is a sugar or a sugar derivative.

12.-14. (canceled)

15. The pharmaceutical composition of claim 10, wherein the excipient is a compound with a hydrophobic component and a PEG or polypropylene glycol component.

16.-19. (canceled)

20. The pharmaceutical composition of claim 10, wherein the pharmaceutical composition comprises two or more excipients.

21.-23. (canceled)

24. The pharmaceutical composition of claim 1, wherein niclosamide comprises from about 0.1% w/w to about 5% w/w of the pharmaceutical composition.

25.-27. (canceled)

28. The pharmaceutical composition of claim 1, wherein the protein comprises from about 40% w/w to about 95% w/w of the pharmaceutical composition.

29.-51. (canceled)

52. The pharmaceutical composition according to claim 1, wherein the pharmaceutical composition is formulated for administration via inhalation.

53. (canceled)

54. The pharmaceutical composition of claim 1, wherein the aerosol pharmaceutical composition has been formulated into an inhaler.

55.-95. (canceled)

96. The pharmaceutical composition of claim 1, wherein the pharmaceutical composition is formulated for nasal administration as a nasal pharmaceutical composition.

97.-124. (canceled)

125. A method of preparing a pharmaceutical composition of claim 1 comprising:
(A) admixing niclosamide and the protein with a solvent to obtain a pharmaceutical mixture;
(B) subjecting the pharmaceutical mixture to spray drying to obtain the pharmaceutical composition.

126. The method of claim 125, wherein the pharmaceutical mixture further comprises an excipient.

127. The method of claim 126, wherein the pharmaceutical mixture comprises two excipients.

128.-139. (canceled)

140. The method of claim 125, wherein the spray drying comprises an inlet temperature from about 80° C. to about 180° C.

141.-147. (canceled)

148. The method claim 125, wherein the spray drying comprises a feed flow rate from about 0.05 mL/min to about 50 mL/min.

149.-153. (canceled)

154. A method of treating a disease or disorder in a patient comprising administering a pharmaceutical composition of claim 1 to the patient in a therapeutically effective amount.

155.-186. (canceled)

187. A method of reducing lung inflammation in a patient comprising administering a pharmaceutical composition according of claim 1 to the patient in a therapeutically effective amount.

188.-193. (canceled)
